SEQUENCE LISTING (36 sequences)

SEQ ID NO: 1 - DNA, 24 bp, Artificial Sequence
  Synthetic forward primer for FMO1
gcaaaacccaacctgttctctatg

SEQ ID NO: 2 - DNA, 20 bp, Artificial Sequence
  Synthetic reverse primer for FMO1
gagcatgggccaaagaagac

SEQ ID NO: 3 - DNA, 22 bp, Artificial Sequence
  Synthetic forward primer for ADH1B
cctgacgttttgaggcaataga

SEQ ID NO: 4 - DNA, 23 bp, Artificial Sequence
  Synthetic reverse primer for ADH1B
cctagctgttgctccagatcttg

SEQ ID NO: 5 - DNA, 16 bp, Artificial Sequence
  Synthetic forward primer for STEAP4
acctttggccccaacc

SEQ ID NO: 6 - DNA, 23 bp, Artificial Sequence
  Synthetic reverse primer for STEAP4
gggaaggacagaaggagaacttg

SEQ ID NO: 7 - DNA, 24 bp, Artificial Sequence
  Synthetic forward primer for DCAMKL1
accacagcacaaagtaactgaact

SEQ ID NO: 8 - DNA, 23 bp, Artificial Sequence
  Synthetic reverse primer for DCAMKL1
tcaactaagtccatcagcagagc

SEQ ID NO: 9 - DNA, 17 bp, Artificial Sequence
  Synthetic forward primer for APOE
ccttggcctggcatcct

SEQ ID NO: 10 - DNA, 17 bp, Artificial Sequence
  Synthetic reverse primer for APOE
ggagccgactggccaat

SEQ ID NO: 11 - DNA, 24 bp, Artificial Sequence
  Synthetic forward primer for SVEP1
gaatgcagattggttcttcacaga

SEQ ID NO: 12 - DNA, 19 bp, Artificial Sequence
  Synthetic reverse primer for SVEP1
cgcccaaatgcttgttcct

SEQ ID NO: 13 - DNA, 20 bp, Artificial Sequence
  Synthetic forward primer for DEPDC1
ggcgctgacagacctatgga

SEQ ID NO: 14 - DNA, 24 bp, Artificial Sequence
  Synthetic reverse primer for DEPDC1
tgctcgaaaagatgtggtaacttc

SEQ ID NO: 15 - DNA, 24 bp, Artificial Sequence
  Synthetic forward primer for hFLEG1
cagcggctgatagagaagtacaac

SEQ ID NO: 16 - DNA, 20 bp, Artificial Sequence
  Synthetic reverse primer for hFLEGC1
gtaggtcagcgtggccattt

SEQ ID NO: 17 - DNA, 18 bp, Artificial Sequence
  Synthetic forward primer for ESM1
cggtggactgccctcaac

SEQ ID NO: 18 - DNA, 20 bp, Artificial Sequence
  Synthetic reverse primer for ESM1
cgtcgagcactgtcctcttg

SEQ ID NO: 19 - DNA, 22 bp, Artificial Sequence
  Synthetic forward primer for TOME-1
attgcacggacacctatgaaga

SEQ ID NO: 20 - DNA, 27 bp, Artificial Sequence
  Synthetic reverse primer for TOME-1
cagtttcaaatacttcactcagctgtt

SEQ ID NO: 21 - DNA, 17 bp, Artificial Sequence
  Synthetic forward primer for THBD
tgtccgcagcgctgtgt

SEQ ID NO: 22 - DNA, 21 bp, Artificial Sequence
  Synthetic reverse primer for THBD
ggtactcgcagttggctctga

SEQ ID NO: 23 - DNA, 22 bp, Artificial Sequence
  Synthetic forward primer for CCL20
agttgtctgtgtgcgcaaatcc

SEQ ID NO: 24 - DNA, 24 bp, Artificial Sequence
  Synthetic reverse primer for CCL20
atgtgcaagtgaaacctccaaccc

SEQ ID NO: 25 - DNA, 25 bp, Artificial Sequence
  Synthetic forward primer for IGFBP3
tacagtgcgcacaggctttatcgag

SEQ ID NO: 26 - DNA, 26 bp, Artificial Sequence
  Synthetic reverse primer for IGFBP3
cgcccttgtttcagaaatgacaccac

SEQ ID NO: 27 - DNA, 24 bp, Artificial Sequence
  Synthetic forward primer for IVL
aaataaccacccgcagtgtccaga

SEQ ID NO: 28 - DNA, 26 bp, Artificial Sequence
  Synthetic reverse primer for IVL
gtagagggacagagtcaagttcacag

SEQ ID NO: 29 - DNA, 23 bp, Artificial Sequence
  Synthetic forward primer for SEMA5B
agccttgccctcaatgcacgaaa

SEQ ID NO: 30 - DNA, 26 bp, Artificial Sequence
  Synthetic reverse primer for SEMA5B
aagcaggtctcagccaacaactctgt

SEQ ID NO: 31 - DNA, 23 bp, Artificial Sequence
  Synthetic forward primer for TSRC1
tgtaacagccaaccctgcagcca

SEQ ID NO: 32 - DNA, 23 bp, Artificial Sequence
  Synthetic reverse primer for TSRC1
acatgtgcgcaagagcggcaaca

SEQ ID NO: 33 - DNA, 24 bp, Artificial Sequence
  Synthetic forward primer for SEZ6L2
aaactggaagtgacccagaccaca

SEQ ID NO: 34 - DNA, 26 bp, Artificial Sequence
  Synthetic reverse primer for SEZ6L2
agggactttccctgaagcttggtgta

SEQ ID NO: 35 - DNA, 24 bp, Artificial Sequence
  Synthetic forward primer for CEBPA
ttgcctaggaacacgaagcacgat

SEQ ID NO: 36 - DNA, 26 bp, Artificial Sequence
  Synthetic reverse primer for CEBPA
cgcacattcacattgcacaaggcact

(19) United States
(12) Patent Application Publication
(10) Pub. No.: US 2010/0291580 A1
(43) Pub. Date: Nov. 18, 2010

(54) BIOMARKERS FOR TRICHOGENICITY

(75) Inventors: Satish Parimoo, Bridgewater, NJ (US); Honghua Yang, Cherry Hill, NJ (US); Ying Homan, Ambler, PA (US); Wei Chen, Havertown, PA (US); Ying Zheng, West Chester, PA (US); Kurt Stenn, Princeton, NJ (US)

Correspondence Address: Pabst Patent Group LLP, 1545 PEACHTREE STREET NE, SUITE 320, ATLANTA, GA, 30309, US

(73) Assignee: Aderans Research Institute, Inc

(21) Appl. No.: 12/808,623

(22) PCT Filed: Dec. 18, 2008
(86) PCT No.: PCT/US08/87513

§ 371 Date: Jun. 16, 2010

Related U.S. Application Data
(60) Provisional application No. 61/014,913, filed on Dec. 19, 2007.
(60) Provisional application No. 61/021,677, filed on Jan. 17, 2008.

(51) Int. Cl. C12Q 1/68; G01N 33/566; C12N 15/79

(52) U.S. Cl. 435/6; 435/7.21; 435/375

(57) ABSTRACT

Biomarkers for identifying trichogenic cells have been identified. The biomarkers include microRNA as wells as mRNA and proteins. Certain biomarkers are upregulated in trichogenic cells compared to non-trichogenic cells; whereas, other biomarkers are down-regulated in trichogenic cells compared to non-trichogenic cells. The cells can be dermal cells, epidermal cells, or a combination thereof. Preferably the cells are mammalian, more preferably the cells are human. One embodiment provides a method for selecting trichogenic cells by assaying the cells for expression of one or more biomarkers for trichogenicity, and selecting the cells having increased expression of the one or more biomarkers relative to a control, wherein increased expression of the a biomarker in the cells is indicative of trichogenicity. Preferably, the one or more biomarkers are selected from the group consisting of hsa-miR-200c, hsa-miR-205, hsa-miR-200a*, hsa-miR-200a, hsa-miR-141, hsa-miR-182, DEPDC1, hFLEG1, ESM1, TOME-1, THBD and combinations thereof.

### FIELD OF THE INVENTION

Aspects of the invention are generally directed to biomarkers for identifying trichogenic cells and methods of use thereof.

### BACKGROUND OF THE INVENTION

Hair loss or alopecia is a common problem in both males and females regardless of their age. There are several types of hair loss, such as androgenetic alopecia, alopecia greata, telogen effluvium, hair loss due to systemic medical problems, e.g., thyroid disease, adverse drug effects and nutritional deficiency states as well as hair loss due to scalp or hair trauma, discoid lupus erythematosus, lichen planus and structural shaft abnormalities. (Hogan and Chamberlain, South Med J, 93(7):657-62 (2000)). Androgenetic alopecia is the most common cause of hair loss, affecting about 50% of individuals who have a strong family history of hair loss. Androgenetic alopecia is caused by three interdependent factors: male hormone dihydrotestosterone (DHT), genetic disposition and advancing age. DHT causes hair follicles to degrade and further shrink in size, resulting in weak hairs. DHT also shortens the anagen phase of the hair follicle growing cycle. Over time, more hairs are shed and hairs become thinner.

Possible options for the treatment of alopecia include hair prosthesis, surgery and topical/oral medications. (Hogan & Chamberlain, 2000; Bertolino, J Dermatol, 20(10):604-10 (1993)). While drugs such as minoxidil, finasteride and dutasteride represent significant advances in the management of male pattern hair loss, the fact that their action is temporary and the hairs are lost after stopping therapy continues to be a major limitation (Bouhanna, Dermatol Surg, 28:136-42 (2002); Avram, et al., Dermatol Surg, 28:894-900 (2002)). In view of this, surgical hair restoration and tissue engineering may be the only permanent methods of treating pattern baldness. The results from surgical hair transplantation can vary and early punch techniques often resulted in a highly unnatural “doll hair look” or “paddy field look” over the recipient area. Although advances have been made in surgical hair transplantation, for example, using single follicle hair grafts with 1 mm punches, the procedures are time consuming and costly and most important, the number of donor follicles on a given patient is limited.

Tissue engineering to treat hair loss includes transplanting cells into an area to induce hair follicle formation and subsequent hair shaft formation.

Theoretically, this simple but effective method of tissue engineering may be employed to treat hair loss due to a variety of diseases, syndromes, and injuries and may provide significant insights into tissue and organ engineering. Hair follicle induction and growth involves active and continuous epithelial and mesenchymal interactions (Sterm & Paus, Physiol Reviews, 81:449-494, (2001)). In the embryo, the first hair follicles grow from a thickening of the primitive epidermis by signals arising from dermal cells. Early studies (Cohen, J Embryol Exp Morphol, 9:117-127 (1961)) using adult rodent hair follicles showed that the dissected deep mesenchymal portion of the hair follicle, the follicular or dermal papilla, when implanted under adult epidermis, will induce new hair follicles. This powerful inductive property is ascribed to a unique property of the cells in the papilla and about the base of the follicle—the dermal sheath (McElwee et al., J Invest Dermatol, 121:1267-1275 (2003)).

Dermal papillae cells from adult rat vibrissae have been implanted into vibrissae from which the lower half, including the dermal papillae, had been removed to promote formation of new hair follicles. Dermal papillae cells can be implanted into adult skin and will induce the formation of new hair follicles from undifferentiated epidermis. The induced hair follicles retain morphologic and hair cycle characteristics of the donor hair follicle dermal papilla (Reynolds and Jahoda, Development, 115:587-593 (1992)). Dermal papillae cells may also be placed in culture to increase cell numbers, which may then be implanted to induce more hair follicle development (Jahoda et al., Nature, 311:560-562 (1984)).

Not all cells obtained from grafts of hair follicles are capable of inducing new hair follicle formation. Much work has been conducted isolating, culturing and characterizing inductive dermal cells from the papilla and sheath (Jahoda, et al., Nature, 311:560-562 (1984); McElwee, et al., 2003; Sleeman, et al., Genomics, 69:214-224 (2000); Rutberg, et al., J Invest Dermatol, 126:2583-259 (2006)). McElwee, et al. discloses that alkaline phosphatase expression can be used as a simple marker to identify mesenchyme derived cells with hair follicle inductive abilities. Unfortunately, alkaline phosphatase is expressed in many different types of cells including liver, bile duct, kidney, bone, and placenta. Biomarkers are needed to distinguish hair follicle inductive cells from non-inductive cells and thus can be used to sort cells.

With the elucidation of the genome of several animals, including man, there has been a major effort in research laboratories about the world to characterize isolated cells, organs, and tumors by the genes they express. Work has been published describing genes expressed by epithelial stem cells of the mouse hair follicle (lumbar, T., et al., Science, 303:359-363 (2004); Morris, R. J., et al., Nature Biotechnology, 22:411-417 (2004)) and human hair follicle (Ohyama, M., et al., J Clin Invest, 116:19-22 (2006)). In the case of the mouse, the cells characterized by a panel of molecules also have the ability to form into new follicles. So, implicit in these reports is the description of a signature of expressed genes which characterize trichogenic cells. Because of the difficulty of growing them in vitro or in vivo, the same kind association or correlation has not been made with human hair follicle cells (Ohyama, M., et al., J Clin Invest, 116:19-22 (2006)).

As new follicle formation involves mesenchymal as well as epithelial cells, studies have also addressed the genes expressed by the mesenchymal compartment of the hair follicle. Extensive gene array studies have been made with dermal papilla (e.g., mouse, Rendl, M., et al., PLOS Biology, 3:1910-1924 (2005), WO2006/124356 to Fuchs et al.; rat, Sleeman, M. A., et al., Genomics, 69:214-224 (2000); and human, e.g., Lu, Z. F., Chin Med J, 119:275-281 (2006)., Rutberg, S. E., J Invest Dermatol, 126:2583-2595 (2006)). Few studies have made correlative studies on the genes expressed in trichogenic dermal cells. The laboratory of Zheng (Lu, Z. F., J. Zhejiang University, 33:296-299 (2004); Lu, Z. F., et al. Chin Med J, 119:275-281, 2006)) reported that dermal cells expressing Stem cell factor and endothelin-1 are more likely to be trichogenic. WO2006/124356 to Fuchs et al. claim that dermal papilla cells expressing BMP6 are more trichogenic. Kishimoto's laboratory reported that the dermal papilla cells are more active in medium which stimulated Wnt pathway but they did not correlate gene expression in those cells with trichogenic activity (Kishimoto, J., et al., Genes Dev, 14:1181-1185 (2000)).

While the above studies focused on expressed coded genes, additional studies have looked for the expression of miRNAs in the hair follicle. These studies were stimulated by the great success achieved using miRNA to characterize human cell lineages and cancer types. These studies did not associate miRNA gene expression with trichogenicity, but they did associate the expression of certain miRNAs with the hair follicle (Ryan, D. G., et al., J Invest Dermatol, 126(4):98 Abstr (2006)) as well as the importance of miRNAs to hair follicle growth and cycling in an miRNA processing-enzyme knockout experiment (Yi, R., et al., Nature Genetics, 38:356-362 (2006); Andl, T., et al., Current Biol, 16:1041-1049 (2006)).

Therefore, it is an object of the invention to provide biomarkers for identifying trichogenic cells.

It is another object to provide microRNA biomarkers for trichogenic cells.

It is another object to provide methods for inducing trichogenesis.

It still another object to provide methods for inhibiting trichogenesis.

### SUMMARY OF THE INVENTION

Biomarkers for identifying trichogenic cells have been identified. The biomarkers include microRNA as wells as mRNA and proteins. Certain biomarkers are upregulated in trichogenic cells compared to non-trichogenic cells; other biomarkers are down-regulated in trichogenic cells compared to non-trichogenic cells. The cells can be dermal cells, epidermal cells, or a combination thereof. Preferably the cells are mammalian, more preferably the cells are human.

Trichogenic cells are initially selected by assaying the cells for expression of one or more biomarkers for trichogenicity, and then selected as those cells having increased expression of the one or more biomarkers relative to a control, wherein increased expression of a biomarker in the cells is indicative of trichogenicity. Preferably, the one or more biomarkers are hsa-miR-200c, hsa-miR-205, hsa-miR-200a*, hsa-miR-200a, hsa-miR-141, hsa-miR-182 or combinations thereof. The cells can be assayed for at least two, three, four, five or more biomarkers of trichogenicity. Alternatively, the one or more biomarkers are encoded by genes DEPDC1, hFLEG1, ESM1, TOME-1, or THBD. In yet another embodiment the one or more biomarkers are encoded by SFRS6, LOC400581, HNT, TNFRSF11B, FOSB, C5R1, HIST1H4C, FGF5, MYBL1, FLJ20105, COL13A1, LOC134285, NEK2, TLR2, VEPH1, KIAA0179, ITGA8, STK6, USP13, C21orf56, CDC45L, C10orf3, TMSNB, TTK, PLAUR, CN/H3, DEPDC1B, ZFAND5, GALNT6, DKFZp313A2432, ASPM, EVI2A, ARTS-1, BUB1, NDP, CDC2, KIF11, HCAP-G, C20orf129, CYCS, TOB1, TBXA2R, FLJ11029, DLG7, KIAA1363, MGC34830, ATAD2, KIF4A, KNTC2, TYMS, KIAA0186, WHSC1, TMEM8, FLJ10038, CIGALT1, KCTD4, FUBP1, FLI1, UBB, NSE1, PTPRD, TNFRSF21, CRYZ, DKFZp761D221, LOC283639, LIMD1, WNT5B, LOC157570, LOC401233, Clorf16, HNRPA1, INCENP, RNF175, CD47, RIN3, SEMA4B, OLFML1, EIF4G3, RoXaN, LRRN3, FZD1, LOC644246, CYYR1, LOC440820, ICK, EST1B, CYLD, PREX1, KIAA1462, MYO10, EIF2AK4, HHEX, HGF, LGR5, PTGIS, HRB2, EFHC2, STYK1, ST8SL44, MYNN, or PPP2R2c.

Preferred biomarkers that have decreased expression in trichogenic cells compared to non-trichogenic cells include, but are not limited to, FMO1, ADM1B, STEAP4, DCAMKL1, APOE, SVEP1 and combinations thereof. Additional biomarkers are encoded by of DKFZP434P211, DKFZP434P211, SPOCK, PTGFR, PDE4DIP, FOXO1A, FLJ14834, C9orf13, SERPING1, ABCA8, STXBP6, LOC339290, KCNE4, CXCL14, MMP10, IFI44L, SLC7A2, LIPG, SERPINA3, ACTG2, TMEM49, KIAA0746, TRIB3, DNM3, LOC440684 (LOC440886), EFEMP1, C5orf13, LOC401212, HCA112, ADAMTS2, GALNTL2, LOC654342, RASD1, SIX2, ZNF179, DSIPI, DCN, LOC283788, CDH2, SYTL4, ASNS, CDW92, HES4, RASGRP2, BET1L, CDK5RAP2, SOX4, AGRN, C12orf22, LIG3, PLEKHG2, NFATC1, LOC440885, RPL37A, SDCBP2, STRN3, SCRG1, NOTCH3, CTNNB1, C18orf11, GARP, SLC2A9, EPPK1, HRH1, C10orf47, JAG1, GABRE, RARRES1, HOXA2, GGA2, LOC158160, PCDH9, PCK2, KLF7, LU, AK3//AK3L2, LIN7B, COL12A1, INHBE, VSNL1, CES1, REC14, SUFU, MRPS11, RNF34, DKFZp667B0210, CACNB2, C13orf25 or a combination thereof.

Biomarkers for identifying trichogenic epidermal cells include, but are not limited to, biomarkers encoded by CCL20, IGFBP3, IVL, SEMA5B, TSRC1, SEZ6L2 or CEBPA. Decreased expression of these biomarkers is indicative of trichogenicity in epidermal cells. Upregulated biomarkers of trichogenicity for epidermal cells include, but are not limited to, APCDD1, IGFBP5, DKFZP586H2123, TXNIP, SCN4B, KRT15, MYLK, PLAC2, UGT1A10//UGT1A8//UGT1A7, CXXC5, GATA3, MAP2, MGC13102, C6orf141, AQP3, DR1, DSC1, HOXA2, ABHD6, RRAD, PPAP2C, KIAA1644, NFATC1, AD023, MYLK, FOSL2, 1HPK2, DOC1, KRT1, CYP2S1, NOTCH3, LGALS7, ABLIM1, CBX4, EPHA4, MUC20, TAGLN, SLC28A3, FOXC1, PVRL4, AMT, KCNJ5, MAF, KIFC2, LOC283970, DLX3, IL1R1V, THRA//NR1D1, TMC4, LOC401320, NIP, EPHB3, MYL9, LOC388335, MARS, C9orf750, C9orf16, PRO1073, BIRC4BP, C5orf19, ERBB3, P53AIP1, IL7, ZNF580, C11ORF4, EPS8L1, DKFZP761M1511, GAPDS, GGT1, TEAD3, FAM46B, BTG2, CEBPD, USP52, P8, MGC11335, C2orf24, SYTL1, PKP1, PPT2, FOXO1A, ZNF606, EGFL6, LOC284801, GULP1, NSUN6, AVPR1B, BEX2, AKAP10, PIP5K1A, DUSP8, CXXC5, ACBD4, MED12, MGC40489, MBNL1, IDUA, IL1R2, DAAM1, HIST1H2BG, AADACL1, LPXN, ZFP42, MARCH4, MFAP5, MGC10850, ZNF367, RAB2, MEST, RRM2, CYGB, C6orf62, HINT3, CLDN11, NPEPL1, ZBED2, FEN1,ARHGAP18, DTL, NAV3, DUSP4, DHX29, LY6K, THBS1, DDAH1, MYBL2, TNF, RAB12, CORO1A, ROBO4, ETV5, NRG1, SLC8A1, HIST1H2B1, AMD1, CYP27B1, SLC39A8, Pfs2, CDC25A, NALP2, TAF1B, and DNMTf.

Another method identifies compounds for enhancing the hair-inducing capability of cultured cells. The method includes assaying the level of one or more biomarkers discussed above in the cells in the presence and in the absence of the putative compound and selecting the compound that increases upregulated biomarkers of trichogenicity or down regulates down-regulated biomarkers of trichogenicity.

Cells can also be genetically engineered to enhance trichogenicity by upregulation expression of one or more genes encoding biomarkers that are upregulated in trichogenic cells relative to non-trichogenic cells. Vectors encoding one or more of the disclosed biomarkers can be inserted into cells to increase or decrease the trichogenicity of the cells. One method includes inserting one or more inhibitory nucleic acids that bind to mRNA of a biomarker for trichogenicity into cells obtained from a subject, wherein the biomarker is up-regulated in trichogenic cells relative to non-trichogenic cells.

### BRIEF DESCRIPTION OF THE DRAWINGS

FIG. 1 is a bar graph of average normalized Ct (ΔCt) values for each of the five miRNA markers assayed by qRT-PCR (quantitative real-time PCR) using SYBR®Green detection and miRNA from trichogenic (+) and non-trichogenic (−) dermal cell samples.

FIG. 2 is a bar graph of individual ΔCt values for hsa-miR-205 marker alone from trichogenic (+) and non-trichogenic (−) dermal cell samples. The average ΔCt±SD of (+) and (−) samples are (4.80±1.9) and (10.98±1.2) respectively.

FIG. 3 is a scatterplot of cumulative ΔCt values for three most distinguishing marker combination ((hsa-miR-10b, hsa-miR-200c and hsa-miR-205) from 21 trichogenic (+) and 10 non-trichogenic (−) dermal cell samples. The average ΔCt±SD of (+) and (−) samples are (22.34±3.08) and (35.97±1.93) respectively.

FIG. 4 shows a Box and Whisker Plot of cumulative ΔCt values for three most distinguishing marker combination ((hsa-miR-10b, hsa-miR-200c and hsa-miR-205) from 21 trichogenic (+) and 10 non-trichogenic (−) dermal cell samples. The spread of data is indicated by horizontal bars and the length of notch around the median (vertical bar) represents an approximate 95% CI for the median.

FIG. 5 shows a graphical representation of average normalized Ct (ΔCt) values (Y-axis) for each of the four miRNA markers as well as cumulative (ΔCt) obtained from qRT-PCR (quantitative real-time PCR) using Taqman® detection system and miRNA from bioassay positive and bioassay negative dermal cell samples. Strongly bioassay positive samples (23 in number) are indicated by ++ and moderately/weakly positive (64 in number) are indicated by +.

FIG. 6 shows a scatterplot of cumulative ΔCt values for four marker combination (hsa-miR-141, hsa-miR-182, hsa-miR-200a and hsa-miR-200a*) from 23 strongly positive (++), 64 moderately/weakly positive (+) and negative (−) dermal cell samples. The average ΔCt±SD of samples are: (++13.25±2.89), (+14.13±4.16) and (−24.26±2.57).

FIG. 7 shows a Box and Whisker Plot of cumulative ΔCt values four marker combination (hsa-miR-141, hsa-miR-182, hsa-miR-200a and hsa-miR-200a*) from 23 strongly positive (++), 64 moderately/weakly positive (+) and negative (−) dermal cell samples. The spread of data is indicated by horizontal bars and the length of notch around the median represents an approximate 95% CI for the median.

FIG. 8 shows a graphical representation of average normalized Ct (ΔCt) values (Y-axis) for each of the six mRNA markers that are down-regulated in from bioassay positive dermal cells in contrast to bioassay negative cells as assayed by qRT-PCR (quantitative real-time PCR) using SYBR®Green detection system. Shown in the Figure is also cumulative (ΔCt) data from these six markers. Strongly positive samples (12 in number) are indicated by ++, moderately and weakly positive (16 in number) are indicated by +, and negative by −(2 in number).

FIG. 9 shows a scatterplot of cumulative ΔCt values for six down-regulated mRNA markers from 12 strongly positive (++), 16 moderately/weakly positive (+) and 2 negative (−) dermal cell samples. The average cumulative ΔCt±SD of samples are: (++72.19±5.90), (+54.19±6.21) and (−46.88±3.75).

FIG. 10 shows a Box and Whisker Plot of cumulative ΔCt values of six down-regulated mRNA markers from 12 strongly positive (++), 16 moderately/weakly positive (+) and 2 negative (−) dermal cell samples. The spread of data is indicated by horizontal bars and the length of notch around the median represents an approximate 95% CI for the median.

FIG. 11 shows a graphical representation of average normalized Ct (ΔCt) values (Y-axis) for each of the five mRNA markers that are up-regulated in mRNA from bioassay positive dermal cells in contrast to mRNA from bioassay negative dermal cells as assayed by qRT-PCR (quantitative real-time PCR) using SYBR®Green detection system. Also shown is cumulative (ΔCt) from these markers. Strongly positive samples (12 in number) are indicated by ++, moderately and weakly positive (16 in number) are indicated by +, and negative by −(2 in number).

FIG. 12 shows a scatterplot of cumulative ΔCt values for five up-regulated mRNA markers from 12 strongly positive (++), 16 moderately/weakly positive (+) and 2 negative (−) dermal cell samples. The average cumulative ΔCt±SD of samples are: (++44.98±2.90), (+51.23±2.79) and (−55.19±1.64).

FIG. 13 shows a Box and Whisker Plot of cumulative ΔCt values of five up-regulated mRNA markers from 12 strongly positive (++), 16 moderately/weakly positive (+) and 2 negative (−) dermal cell samples. The spread of data is indicated by horizontal bars and the length of notch around the median represents an approximate 95% CI for the median.

FIG. 14 shows a graphical representation of average normalized Ct (ΔCt) values (Y-axis) for each of the seven mRNA markers that are down-regulated in mRNA from bioassay positive cells in contrast to mRNA from bioassay negative cells as assayed by qRT-PCR (quantitative real-time PCR) using SYBR®Green detection system. Also shown is cumulative (ΔCt) from these seven markers. Strongly positive samples (15 in number) are indicated by (++), moderately and weakly positive (10 in number) are indicated by (+), and 4 negative by (−).

FIG. 15 shows a scatterplot of cumulative ΔCt values for seven down-regulated mRNA markers from 15 strongly positive (++), 10 moderately/weakly positive (+) and 4 negative (−) dermal cell samples. The average ΔCt±SD of samples are: (++62.96±2.91), (+57.51±3.98) and (−49.15±2.16).

FIG. 16 shows Box and Whisker Plot of cumulative ΔCt values seven down-regulated mRNA markers from 15 strongly positive (++), 10 moderately/weakly positive (+) and 4 negative (−) dermal cell samples. The spread of data is indicated by horizontal bars and the length of notch around the median represents an approximate 95% CI for the median.

### DETAILED DESCRIPTION OF THE INVENTION

### I. Definitions

The term “trichogenic cells” refers to cells that induce hair follicle formation. Induction of hair follicles can be direct or indirect. The term “effective amount” refers to an amount of cells needed to induce hair follicle formation.

As used herein the term “isolated” is meant to describe cells that are in an environment different from that in which the cells naturally occur e.g., separated from its natural milieu such as by separating dermal cells from a hair follicle.

The terms “individual”, “host”, “subject”, and “patient” are used interchangeably herein, and refer to a mammal, including, but not limited to, murines, simians, humans, mammalian farm animals, mammalian sport animals, and mammalian pets.

As used herein the term “effective amount” or “therapeutically effective amount” means an amount of cells sufficient to induce hair follicle formation or to induce vellus hair to form terminal hair.

As used herein the term “skin” refers to the outer covering of an animal. In general, the skin includes the epidermis and the dermis.

The term “biomarker” refers to a nucleic acid or protein whose expression levels are indicative of trichogenicity. Certain biomarkers are expressed at higher levels in trichogenic cells compared to non-trichogenic cells. Other biomarkers have reduced expression levels in trichogenic cells compared to non-trichogenic cells.

### II. Biomarkers for Trichogenic Cells

Biomarkers for identifying trichogenic cells are provided. The biomarkers include certain mRNAs or the proteins encoded by the mRNAs as well as microRNAs. In certain embodiments, levels of at least one, two, or even three biomarkers can be used to identify trichogenic cells, preferably dermal or epidermal cells. In trichogenic cells, the biomarker can be at detectable levels relative to nondetectable levels in non-trichogenic cells; at higher levels than non-trichogenic cells, or at levels lower than non-trichogenic cells. The cells are eukaryotic cells, preferably mammalian cells such as human or rodent dermal or epidermal cells. Preferred biomarkers are provided below.

A. MicroRNA Biomarkers of Trichogenicity

It has been discovered that the presence of certain microRNAs in cells is indicative of trichogenicity. MicroRNAs (miRNAs) are small RNA molecules encoded in the genomes of plants and animals. These highly conserved, about 21-mer RNAs regulate the expression of genes by binding to the 3′-untranslated regions (3′-UTR) of specific mRNAs. The miRNAs can be 19, 20, 21, 22, 23, or more contiguous nucleotides.

Although the first published description of an miRNA appeared fifteen years ago (Lee, R. C., et al., Cell, 75: 843-854 (1993)), only in the last two to three years has the breadth and diversity of this class of small, regulatory RNAs been appreciated. A great deal of effort has gone into understanding how, when, and where miRNAs are produced and function in cells, tissues, and organisms. Each miRNA is thought to regulate multiple genes, and since hundreds of miRNA genes are predicted to be present in higher eukaryotes (Lim, L. P., Science, 299: 1540 (2003)) the potential regulatory circuitry afforded by miRNA is enormous. Several research groups have provided evidence that miRNAs may act as key regulators of processes as diverse as early development (Reinhart, B. J., et al., Nature, 403: 901-906 (2000)), cell proliferation and cell death (Brennecke, J., et al., Cell, 113: 25-36 (2003)), apoptosis and fat metabolism (Xu, P., et al., Curr Biol, 13(9): 790-5 (2003)), and cell differentiation (Dostie, J., et al., RNA, 9(2): 180-6 (2003) Erratum in: RNA 9(5): 631-2; Chen, X., Science, 26; 303(5666) (2004)). Other studies of miRNA expression implicate miRNAs in brain development (Krichevsky, A. M., et al., RNA, 9: 1274-1281 (2003)), chronic lymphocytic leukemia (Calin, G. A., et al., Proc. Natl. Acad. Sci. USA., 101: 2999-3004 (2004)), colonic adenocarcinoma (Michael, M. Z., et al., Molecular Cancer Research, 1: 882-91 (2003)), Burkitt's Lymphoma (Metzler, M., et al., Genes Chromosomes Cancer, 2: 167-169 (2004)), and viral infection (Pfeffer, S., et al., Science, 304(5671): 734-6 (2004)) suggesting possible links between miRNAs and viral disease, neurodevelopment, and cancer. There is speculation that in higher eukaryotes, the role of miRNAs in regulating gene expression could be as important as that of transcription factors.

It has now been discovered the miRNAs can be indicators of trichogenicity. One embodiment provides miRNA biomarkers for trichogenicity of human or murine dermal cells including, but not limited to, a miRNA biomarker encoded by hsa-miR-10b, hsa-miR-200c, hsa-miR-205, hsa-miR-10a or hsa-miR-382. Preferred miRNAs are encoded by hsa-miR-10b, hsa-miR-200c, and hsa-miR-205.

Expression levels of the biomarker in trichogenic and non-trichogenic cells can be detected using conventional techniques such as real time PCR. In a real time PCR assay a positive reaction is detected by accumulation of a fluorescent signal. The Ct (cycle threshold) is defined as the number of cycles required for the fluorescent signal to cross the threshold (i.e., exceeds background level). Ct levels are inversely proportional to the amount of target nucleic acid in the sample (i.e., the lower the Ct level the greater the amount of target nucleic acid in the sample). Ct values can be used to calculate the relative difference in expression of biomarkers in samples by using the formula: fold expression=2^{-ΔΔCt}, where AΔCt is the difference in normalized Ct values of the two samples being compared. Thus, the relative expression of hsa-miR-10b, hsa-miR-200c, and hsa-miR-205 in trichogenic cells is greater than the relative expression of these miRNAs in non-trichogenic cells. Non-trichogenic cells can be distinguished from trichogenic cells using the Aderans Hair Patch Assay™ described in Example 1 and in Zheng, Y., J Invest Dermatol, 124: 867-876 (2005). Elevated expression levels of any one of hsa-miR-10b, hsa-miR-200c, and hsa-miR-205 or a combination thereof can be used to identify trichogenic cells, preferably trichogenic dermal cells.

Additional miRNA biomarkers for trichogenicity include, but are not limited to, miRNA biomarkers encoded by hsa-miR-200a*, hsa-miR-200a, hsa-miR-141 and optionally hsa-miR-182. Identification of trichogenic cells can be accomplished by detecting elevated expression of at least one, two, or three of the disclosed miRNA biomarkers as compared to expression levels of these biomarkers in non-trichogenic cells.

B. Messenger RNA/Protein Biomarkers of Trichogenicity

1. Down-Regulated Biomarkers of Trichogenicity

In addition to miRNA biomarkers, mRNA biomarkers or protein biomarkers encoded by specific genes have been identified as biomarkers for trichogenicity in mammalian cells, preferably human or murine dermal cells. mRNA biomarkers for trichogenicity having decreased expression levels compared to non-trichogenic cells include, but are not limited to, mRNA biomarkers encoded by the following genes: FMO1, ADH1B, STEAP4, DCAMKL1, APOE, and SVEP1. Identification of trichogenic cells can be accomplished by detecting decreased expression levels of at least one, two, or three of the disclosed mRNA biomarkers as compared to expression levels of these biomarkers in non-trichogenic cells.

The mRNA biomarkers can vary in size from about 50, 100, 200, 300, 600, 900, or even 1,500 or more nucleotides.

Additional biomarkers that have reduced expression in trichogenic dermal cells as compared to non-trichogenic dermal cells are encoded by the following genes: DKFZP434P211, DKFZP434P211, SPOOK PTGFR, PDE4D1P, FOXO1A, FLJ14834, C9orf13, SERPING1, ABCA8, STXBP6, LOC339290, KCNE4, CXCL14, MMP10, IFI44L, SLC7A2, LIPG, SERPINA3, ACTG2, TMEM49, KIAA0746, TRIB3, DNM3, LOC440684 (LOC440886), EFEMP1, C5orf13, LOC401212, HCA112, ADAMTS2, GALNTL2, LOC654342, RASD1, SIX2, ZNF179, DSIPI, DCN, LOC283788, CDH2, SYTL4, ASNS, CDW92, HES4, RASGRP2, BET1L, CDK5RAP2, SOX4, AGRN, C12orf22, LIG3, PLEKHG2, NFATC1, LOC440885, RPL37A, SDCBP2, STRN3, SCRG1, NOTCH3, CTNNB1, C18orf71, GARP, SLC2A9, EPPK1, HRH1, C10orf47, JAG1, GABRE, RARRES1, HOXA2, GGA2, LOC158160, PCDH9, PCK2, KLF7, LU, AK3//AK3L2, LIN7B, COL12A1, INHBE, VSNL1, CES1, REC14, SUFU, MRPS11, RNF34, DKFZp667B0210, CACNB2, and C13orf25.

It will be appreciated that levels of proteins encoded by the disclosed mRNA biomarkers can be used as biomarkers for trichogenicity. Methods for detecting levels of proteins in a sample are known in the art and include, but are not limited to, mass spectroscopy and immunohistochemical methods including ELISA, Western blot, and immunoprecipitation.

2. Up-regulated Biomarkers of Trichogenicity

mRNA biomarkers of trichogenicity that have elevated expression levels compared to non-trichogenic human or murine dermal cells have also been identified. Preferred mRNA biomarkers having elevated expression include, but are not limited to, DEPDC1, hFLEG1, ESM1, TOME-1, and optionally THBD. Identification of trichogenic cells can be accomplished by detecting increased expression levels of at least one, two, or three of the disclosed mRNA biomarkers as compared to expression levels of these biomarkers in non-trichogenic cells.

Additional biomarkers that are upregulated in trichogenic cells compared to non-trichogenic cells include biomarkers encoded by the following genes: SFRS6, LOC400581, HNT, TNFRSF11B, FOSB, C5R1, HIST1H4C, FGF5, MYBL1, FLJ20105, COL13A1, LOC134285, NEK2, TLR2, VEPH1, KIAA0179, ITGA8, STK6, USP13, C21orf56, CDC45L, C10orf3, TMSNB, TTK, PLAUR, CN/H3, DEPDC1B, ZFAND5, GALNT6, DKFZp313A2432, ASPM, EVI2A, ARTS-1, BUB1, NDP, CDC2, KIF11, HCAP-G, C20orf129, CYCS, TOB1, TBXA2R, FLJ11029, DLG7, KIAA1363, MGC34830, ATAD2, KIF4A, KNTC2, TYMS, KIAA0186, WHSC1, TMEM8, FLJ10038, CIGALT1, KCTD4, FUBP1, FLI1, UBB, NSE1, PTPRD, TNFRSF21, CRYZ, DKFZp761D221, LOC283639, LIMD1, WNT5B, LOC157570, LOC401233, Clorf16, HNRPA1, INCENP, RNF175, CD47, RIN3, SEMA4B, OLFML1, EIF4G3, RoXaN, LRRN3, FZD1, LOC644246, CYYR1, LOC440820, ICK, EST1B, CYLD, PREX1, KIAA1462, MYO10, EIF2AK4, HHEX, HGF, LGR5, PTGIS, HRB2, EFHC2, STYK1, ST8SIA4, MYNN, and PPP2R2c.

Proteins encoded by the disclosed genes can be used as biomarkers for trichogenicity by comparing the levels of the protein in trichogenic cells to levels of the protein in non-trichogenic cells.

3. Down-regulated Biomarkers for Trichogenic Epidermal Cells

Another embodiment provides mRNA/protein biomarkers for identifying trichogenic epidermal cells, preferably, human epidermal cells. These biomarkers include, but are not limited to, biomarkers encoded by the following genes: CCL20, IGFBP3, IVL, SEMA5B, TSRC1, SEZ6L2, and CEBPA. Identification of trichogenic cells can be accomplished by detecting decreased expression levels of at least one, two, or three of the disclosed mRNA/protein biomarkers as compared to expression levels of these biomarkers in non-trichogenic cells.

4. Up-regulated Biomarkers for Trichogenicity of Epidermal Cells

Still another embodiment provides mRNA/protein biomarkers for trichogenic epidermal cells encoded by the following genes: APCDD1, IGFBP5, DKFZP586H2123, TXNIP, SCN4B, KRT15, MYLK, PLAC2, UGT1A10//UGT1A8//UGT1A7, CXXC5, GATA3, MAP2, MGC13102, C6orf141, AQP3, DR1, DSC1, HOXA2, ABHD6, RRAD, PPAP2C, K1AA1644, NFATC1, AD023, MYLK, FOSL2, 1HPK2, DOC1, KRT1, CYP2S1, NOTCH3, LGALS7, ABLIM1, CBX4, EPHA4, MUC20, TAGLN, SLC28A3, FOXC1, PVRL4, AMT, KCNJ5, MAF, KIFC2, LOC283970, DLX3, IL1RN, THRA//NR1D1, TMC4, LOC401320, NIP, EPHB3, MYL9, LOC388335, MARS, C9orf150, C9orf16, PRO1073, BIRC4BP, C5orf19, ERBB3, P53AIP1, IL7, ZNF580, C11ORF4, EPS8L1, DKFZP761M1511, GAPDS, GGT1, TEAD3, FAM46B, BTG2, CEBPD, USP52, P8, MGC11335, C2orf24, SYTL1, PKP1, PPT2, FOXO1A, ZNF606, EGFL6, LOC284801, GULP1, NSUN6, AVPR1B, BEX2, AKAP10, PIP5K1A, DUSP8, CXXC5, ACBD4, MED12, MGC40489, MBNL1, IDUA, IL1R2, DAAM1, HIST1H2BG, AADACL1, LPXN, ZFP42, MARCH4, MFAP5, MGC10850, ZNF367, RAB2, MEST, RRM2, CYGB, C6orf62, HINT3, CLDN11, NPEPL1, ZBED2, FEN1,ARHGAP18, DTL, NAV3, DUSP4, DHX29, LY6K, THBS1, DDAH1, MYBL2, TNF, RAB12, CORO1A, ROBO4, ETV5, NRG1, SLC8A1, HIST1H2BI, AMD1, CYP27B1, SLC39A8, Pfs2, CDC25A, NALP2, TAF1B, and DNMT2. Identification of trichogenic cells can be accomplished by detecting increased expression levels of at least one, two, or three of the disclosed mRNA/protein biomarkers as compared to expression levels of these biomarkers in non-trichogenic cells.

5. Combinations of Biomarkers

Combinations of the disclosed biomarkers can be used to distinguish trichogenic cells from non-trichogenic cells. In one embodiment, combinations of miRNA biomarkers with mRNA/protein biomarkers can be used. Sets of biomarkers that are expressed in trichogenic cells or have increased expression in trichogenic cells can be used in any combination. Thus, one embodiment provides miRNA biomarkers in combination with mRNA/protein biomarkers wherein the mRNA/protein biomarkers have increased expression in trichogenic cells relative to non-trichogenic cells. Another embodiment provides miRNA biomarkers in combination with mRNA/protein biomarkers wherein the mRNA/protein biomarkers have reduced or non-detectable expression in trichogenic cells relative to non-trichogenic cells. In another embodiment, combinations of microRNA biomarkers are used. In yet another embodiment, combinations of mRNA/protein biomarkers are used to identify trichogenic cells.

Preferably, levels of one, two, three or more of the following biomarkers can be determined to identify trichogenic cells: hsa-miR-200a*, hsa-miR-200a, hsa-miR-141, hsa-miR-200c, hsa-miR-205, DEPDC1, hFLEG1, ESM1, TOME-1 and THBD.

### III. Methods for Using Biomarkers for Trichogenic Cells

A. Identification of Trichogenic Cells

One or more of the disclosed biomarkers can be used to identify trichogenic cells. Generally, cells are harvested from an animal, for example a mouse or human. The cells can be autologous or allogenic. Tissue, preferably scalp tissue, is obtained from a subject and processed to obtain dissociated cells using techniques known in the art. The cells are a mixed population of cells containing both trichogenic cells and non-trichogenic cells. In some embodiments the mixed population of cells includes both dermal and epidermal cells. The dermal and epidermal cells can be trichgenic or non-trichogenic or a combination thereof.

Trichogenic cells in a mixed population of cells are identified by assaying the cells for one or more of the biomarkers described above. Methods for identifying nucleic acid or protein biomarkers are known in the art. Quantitative Real-Time PCR, flow cytometry and immunological techniques are preferred.

In one embodiment a population of cells enriched for expression of one or more trichogenic biomarkers is obtained by cell sorting using CELLection™ Biotin Binder Kit. Both direct and indirect methods can be employed. Basically, the biotinylated anti-biomarker antibody is added to the cell sample at 1 μg per 1 million cells (indirect method) or added to streptavidin coated beads at 2 μg/25 ul beads (direct method) and incubate at 4° C. overnight. The streptavidin coated beads can be moved using a magnet. Next, the streptavidin coated beads and cell sample are mixed together so the biomarker positive cells attach to the streptavidin coated beads through the biotinylated anti-biomarker antibody. The bead-bound-cells are then separated from other cells by a magnet. The biomarker positive cells are then digested from the magnetic beads after incubating with DNase I at room temperature for 15 minutes. The beads are then removed using magnets.

In another embodiment, biomarker expression is detected by Guava Analyzer. Briefly, cells are first incubated with a Phycoerythrin conjugated anti-biomarker antibody at 4° C. for half an hour. Then the cells are washed two times with Dulbecco's Phosphate Buffered Saline (DPBS) with bovine serum albumin (0.1% BSA) plus antibiotic (clindamycin, actinomycin, streptomycin). Biomarker expression level is measured by GUAVA Analyzer.

B. Screening for Compounds that Modulate Trichogenicity

Methods for identifying modulators of trichogenicity can be accomplished using well known techniques and reagents. In some embodiments, the assays can include random screening of large libraries of test compounds. Alternatively, the assays may be used to focus on particular classes of compounds suspected of modulating trichogenicity.

Assays can include determinations of the disclosed biomarker gene expression, protein expression, protein activity, or binding activity. Other assays can include determinations of biomarker nucleic acid transcription or translation, for example mRNA levels, miRNA levels, mRNA stability, mRNA degradation, transcription rates, and translation rates.

In one embodiment, the identification of a modulator of trichogenicity is based on the function of the biomarker in the presence and absence of a test compound. The test compound or modulator can be any substance that alters or is believed to alter the function of the biomarker. Typically, a modulator will be selected that reduces, eliminates, or inhibits trichogenicity as determined using the assays described herein. Alternatively, modulators that increase or enhances trichogenicity are selected.

One exemplary method includes contacting a biomarker with at least a first test compound, and assaying for an interaction between the biomarker and the first test compound with an assay. The assaying can include determining biological function of the biomarker including expression and bioavailability of the biomarker.

Specific assay endpoints or interactions that may be measured in the disclosed embodiments include assaying for biomarker nucleic acid expression or levels of biomarker protein. These assay endpoints may be assayed using standard methods such as FACS, FACE, ELISA, Northern blotting and/or Western blotting. Moreover, the assays can be conducted in cell free systems, in isolated cells, genetically engineered cells, immortalized cells, or in organisms and transgenic animals.

Other screening methods include using labeled biomarkers to identify a test compound. Biomarkers can be labeled using standard labeling procedures that are well known and used in the art. Such labels include, but are not limited to, radioactive, fluorescent, biological and enzymatic tags.

Another embodiment provides a method for identifying a modulator of trichogenicity by determining the effect a test compound has on the expression of one or more biomarkers in cells. For example isolated cells or whole organisms expressing one or more biomarkers for trichogenicity can be contacted with a test compound. Gene expression can be determined by detecting biomarker protein expression or mRNA transcription or translation. Suitable cells for this assay include, but are not limited to, immortalized cell lines, primary cell culture, or cells engineered to express the biomarker. Compounds that modulate the expression of the biomarker in particular that enhance or increase the expression or bioavailability of biomarker can be selected. Alternatively, compounds that decrease or reduce biomarker expression or activity can be selected.

One example of a cell free assay is a binding assay. While not directly addressing function, the ability of a modulator to bind to a target molecule, for example a nucleic acid encoding a biomarker, in a specific fashion is strong evidence of a related biological effect. Such a molecule can bind to a biomarker nucleic acid and modulate expression of the biomarker for example up-regulate expression of the biomarker. The binding of a molecule to a target may, in and of itself, be inhibitory, due to steric, allosteric or charge—charge interactions or may downregulate or inactivate the biomarker. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

In one embodiment a transgenic cell is used to produce, typically, over produce the biomarker. The transgenic cell can include an expression vector encoding the biomarker. The introduction of DNA into a cell or a host cell is well known technology in the field of molecular biology and is described, for example, in Sambrook et al., Molecular Cloning 3rd Ed. (2001). Methods of transfection of cells include calcium phosphate precipitation, liposome mediated transfection, DEAE dextran mediated transfection, electroporation, ballistic bombardment, and the like. Alternatively, cells may be simply transfected with the disclosed expression vector using conventional technology described in the references and examples provided herein. The host cell can be a prokaryotic or eukaryotic cell, or any transformable organism that is capable of replicating a vector and/or expressing a heterologous gene encoded by the vector. Numerous cell lines and cultures are available for use as a host cell, and they can be obtained through the American Type Culture Collection (ATCC), which is an organization that serves as an archive for living cultures and genetic materials (www.atcc.org).

A host cell can be selected depending on the nature of the transfection vector and the purpose of the transfection. A plasmid or cosmid, for example, can be introduced into a prokaryote host cell for replication of many vectors. Bacterial cells used as host cells for vector replication and/or expression include DH5α, JM109, and KCB, as well as a number of commercially available bacterial hosts such as SURE® Competent Cells and SOLOPACK™ Gold Cells (STRATAGENE, La Jolla, Calif.). Alternatively, bacterial cells such as E. coli LE392 could be used as host cells for phage viruses. Eukaryotic cells that can be used as host cells include, but are not limited to, yeast, insects and mammals. Examples of mammalian eukaryotic host cells for replication and/or expression of a vector include, but are not limited to, HeLa, NIH3T3, Jurkat, 293, Cos, CHO, Saos, and PC12. Examples of yeast strains include YPH499, YPHS500 and YPHS501. Many host cells from various cell types and organisms are available and would be known to one of skill in the art. Similarly, a viral vector may be used in conjunction with either an eukaryotic or prokaryotic host cell, particularly one that is permissive for replication or expression of the vector. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

In vivo assays involve the use of various animal models, including non-human transgenic animals that have been engineered to have specific defects, or carry markers that can be used to measure the ability of a test compound to reach and affect different cells within the organism. Due to their size, ease of handling, and information on their physiology and genetic make-up, mice are a preferred embodiment, especially for transgenic animals. However, other animals are suitable as well, including C. elegans, rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more test compounds are administered to an animal, and the ability of the test compound(s) to alter trichogenicity, as compared to a similar animal not treated with the test compound(s), identifies a modulator. Other embodiments provide methods of screening for a test compound that modulates the function of the biomarker. In these embodiments, a representative method generally includes the steps of administering a test compound to the animal and determining the ability of the test compound to promote or inhibit trichogenicity.

Treatment of these animals with test compounds will involve the administration of the compound, in an appropriate form, to the animal. Administration will be by any route that could be utilized for clinical or non-clinical purposes, including, but not limited to, oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

C. Modulating Trichogenicity

1. Inducing or Inhibiting Expression of Biomarkers for Trichogenicity Epigenetically

Methods for inducing trichogenicity in cells are also provided. Typically a cell, preferably a dermal cell, epidermal cell, or a combination thereof is contacted with an agonist or antagonist of a biomarker that is up-regulated in trichogenic cells compared to non-trichogenic cells. The agonist induces expression of the biomarker or induces biological activity of the biomarker relative to controls leading to an increase in trichogenicity. The antagonist inhibits expression of the biomarker or inhibits biological activity of the biomarker relative to controls leading to a decrease in trichogenicity. Suitable up-regulated biomarkers are described above. Preferred miRNA biomarkers include one or more of hsa-miR-200a*, hsa-miR-200a, hsa-miR-141, hsa-miR-182. hsa-miR-200c, and hsa-miR-205. Preferred up-regulated biomarkers for trichogenicity include, but are not limited to protein or mRNA biomarkers encoded by a gene selected from the group consisting of DEPDC1, hFLEG1, ESM1, TOME-1, THBD and combinations thereof.

Alternatively, a subject's cells are transfected with nucleic acids encoding one more biomarkers that are up-regulated in trichogenic cells relative to non-trichogenic cells. The expression of the biomarkers can be modulated by using strong promoters to overexpress the biomarker, or using inducible promoters to control when the biomarkers are expressed. Strong promoters and inducible promoters are known in the art.

Nucleic acids encoding the up-regulated biomarker may also be used in gene therapy. In gene therapy applications, genes are introduced into cells in order to achieve in vivo synthesis of a therapeutically effective genetic product, for example, protein or nucleic acid that promotes trichogenicity. “Gene therapy” includes both conventional gene therapy where a lasting effect is achieved by a single treatment, and the administration of gene therapeutic agents, which involves the one time or repeated administration of a therapeutically effective DNA or mRNA. Any of a variety of techniques known in the art may be used to introduce nucleic acids to the relevant cells.

The nucleic acids or oligonucleotides may be modified to enhance their uptake, e.g., by substituting their negatively charged phosphodiester groups by uncharged groups. For review of gene marking and gene therapy protocols see Anderson et. al., Science 256:808-813 (1992).

In another embodiment, cells are contacted with antagonists of up-regulated biomarkers of trichogenicity. Antagonists inhibit or reduce the expression or biological activity of the up-regulated biomarkers of trichogenicity. Suitable antagonists include, but are not limited to, inhibitory nucleic acids such as ribozymes, triplex-forming oligonucleotides (TFOs), antisense DNA, siRNA, and microRNA specific for nucleic acids encoding the biomarkers.

Useful inhibitory nucleic acids include those that reduce the expression of RNA encoding the biomarkers by at least 20%, 30%, 40%, 50%, 60%, 70%, 80%, 90% or 95% compared to controls. Expression of the biomarkers can be measured by methods well know to those of skill in the art, including northern blotting and quantitative polymerase chain reaction (PCR).

Inhibitory nucleic acids and methods of producing them are well known in the art. siRNA design software is available for example at http://i.cs.hku.hk/˜sirna/software/sirna.php. Synthesis of nucleic acids is well known see for example Molecular Cloning: A Laboratory Manual (Sambrook and Russel eds. 3^{rd} ed.) Cold Spring Harbor, N.Y. (2001). The term “siRNA” means a small interfering RNA that is a short-length double-stranded RNA that is not toxic. Generally, there is no particular limitation in the length of siRNA as long as it does not show toxicity. “siRNAs” can be, for example, 15 to 49 bp, preferably 15 to 35 bp, and more preferably 21 to 30 by long. Alternatively, the double-stranded RNA portion of a final transcription product of siRNA to be expressed can be, for example, 15 to 49 bp, preferably 15 to 35 bp, and more preferably 21 to 30 by long. The siRNA can be at least 19, 20, 21, 22, 23, 24, or 25 contiguous nucleotides in length. The double-stranded RNA portions of siRNAs in which two RNA strands pair up are not limited to the completely paired ones, and may contain nonpairing portions due to mismatch (the corresponding nucleotides are not complementary), and bulge (lacking in the corresponding complementary nucleotide on one strand). Nonpairing portions can be contained to the extent that they do not interfere with siRNA formation. The “bulge” used herein preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, the double-stranded RNA region of siRNAs in which two RNA strands pair up may contain both bulge and mismatched, which sum up to, preferably 1 to 7, more preferably 1 to 5 in number.

The terminal structure of siRNA may be either blunt or cohesive (overhanging) as long as siRNA can silence, reduce, or inhibit the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 by double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the siRNA is not necessarily the cut off structure at both ends as described above, and may have a stem-loop structure in which ends of one side of double-stranded RNA are connected by a linker RNA. The length of the double-stranded RNA region (stem-loop portion) can be, for example, 15 to 49 bp, preferably 15 to 35 bp, and more preferably 21 to 30 by long. Alternatively, the length of the double-stranded RNA region that is a final transcription product of siRNAs to be expressed is, for example, 15 to 49 bp, preferably 15 to 35 bp, and more preferably 21 to 30 by long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA or viral RNA, or an artificial RNA molecule.

miRNAs are produced by the cleavage of short stem-loop precursors by Dicer-like enzymes; whereas, siRNAs are produced by the cleavage of long double-stranded RNA molecules. miRNAs are single-stranded, whereas siRNAs are double-stranded.

Methods for producing siRNA are known in the art. Because the sequences for fibronectin or aggrecan known, one of skill in the art could readily produce siRNAs that downregulate fibronectin or aggrecan expression using information that is publicly available.

### EXAMPLES

### Example 1

### Bioassay for Trichogenicity Evaluation

Aderans Hair Patch Assay™

Trichogenic activity of populations of dermal cells was determined by the Aderans Hair Patch Assay™ (Zheng, Y., J Invest Dermatol, 124: 867-876 (2005)). In this assay dissociated dermal and epidermal cells are implanted into the dermis or the subcutis of an immunoincompetent mouse. Using mouse newborn skin cells, new hair follicles typically form in this assay within 8 to 10 days. The newly formed follicle manifests normal hair shafts, mature sebaceous glands, and a natural hair cycle. Although normal cycling hair follicles are formed in this assay, the assay primarily measures the ability of cells or combinations of cells to form new follicles. Mouse dermal cells were assayed in conjunction with mouse neonatal epidermal cells as described (Zheng et al. 2005).

Results

Cultured human dermal cells or epidermal cells derived from scalp were assayed for their trichogenicity (hair inducing ability) by Aderans Hair Patch Assay™ in nude mice. Positive human cultured samples generated hair in the bioassay. Negative samples did not.

### Example 2

### MicroRNA Biomarkers of Trichogenicity

RNA Isolation

Total RNA or microRNA (miRNA) enriched small RNA fraction were isolated from human scalp derived dermal cells or epidermal cells cultured in serum-free growth media at culture passage P-1 using commercially available kits (Ambion) for RNA isolation. RNA samples were used for DNA microarrays to identify candidate markers for trichogenicity (hair-inducing capability) that were further evaluated by Quantitative Real-Time PCR (qRT-PCR).

Gene Profiling

Gene profiles were obtained using total RNA from trichogenic (bioassay positive) or non-trichogenic (bioassay negative) cultured human cells using Affymetrix gene arrays (Human U133Plus 2.0—Whole Genome). RNA from mouse cells were gene profiled for differentially regulated genes between trichogenic and non-trichogenic samples using Affymetrix arrays MOE 430A and MOE 430B. MicroRNA gene candidates were identified by microRNA profiling using mirVana™ miRNA Bioarray 1566 as well as multiplex RT-PCR.

Reverse Transcription and Real-Time PCR

cDNA was synthesized from RNA samples by reverse transcription followed by individual marker expression analysis by Quantitative Real-Time PCR (qRT-PCR). qRT-PCR reactions were set up with either total RNA for mRNA markers or miRNA enriched fraction for miRNA markers using reagents from commercially available kits for reverse transcription and PCR. miRNA markers were evaluated by qRT-PCR using either Taqman® detection system with RNU43 as endogenous control for data normalization or SYBR® Green detection system and 5sRNA as endogenous control. miRNA markers were purchased from either Applied Biosystems (Taqman® based markers) or Ambion (SYBR®Green based markers). Oligonucleotide primers for mRNA markers, including GAPDH as endogenous control, were custom synthesized based on genome sequence available from public domain database (NCBI).

For miRNA markers using SYBR® ® Green detection in qRT-PCR, reverse transcription reactions were set up in 10 μl volume containing 20 ng of miRNA with reagents from mirVana™ qRT-PCR miRNA Detection Kit (Ambion) following vendor's instructions. Samples were incubated for 30 min at 37° C., then for 10 min at 95° C. PCR was carried out in 25 μl volume using SYBR® ® Green PCR Reagents (Applied Biosystems), except mirVana™ qRT-PCR Primers and SuperTaq™ Polymerase were from Ambion. Thermal cycling conditions for PCR amplification of miRNA target sequences include: initial denaturation of 95° C. for 3 minutes followed by 35 cycles of denaturation 95° C. for 15 seconds, annealing and extension at 60° C. for 30 seconds.

For miRNA markers using Taqman® detection in qRT-PCR, reverse transcription reactions were set up in 7.5 μl volume containing 100 ng of total RNA or 10 ng of miRNA with reagents from Taqman® microRNA RT Kit (Applied Biosystems) following the vendor's instructions. Samples were incubated for 30 min at 16° C., then for 30 min at 42° C., followed by 5 min at 85° C. PCR was carried out in 25 μl volume using 1.7 ul of reverse transcription product, Taqman® Universal Master Mix (Applied Biosystems) following the vendor's instructions. PCR amplification was carried out in a Real-Time PCR machine (Applied Biosystems) using a thermocycling program of initial denaturation at 95° C. for 10 min (1 cycle), followed by 40 cycles of denaturation at 95° C. for 15 sec, annealing and extension at 60° C. for 60 sec.

For mRNA markers, reverse transcription reactions were set up in 50 μl volume containing 1 μg total RNA of miRNA with random hexamers, MultiScribe™ Reverse Transcriptase and other reagents from Taqman Reverse Transcription (Applied Biosystems) following the vendor's instructions. Samples were incubated for 10 min at 25° C., 30 min at 48° C., 5 min at 85° C. PCR was carried out in 25 μl volume using 2.5 μl of reverse transcription product, AmpliTaq Gold and reagents from SYBR® Green PCR Core Reagents (Applied Biosystems). PCR amplification was carried out in Real-Time PCR machine (Applied Biosystems) using a thermocycling program of initial denaturation at 95° C. for 10 min (1 cycle), followed by 40 cycles of denaturation at 95° C. for 15 sec, annealing at 58° C. for 32 sec and extension at 72° C. for 32 sec.

Results

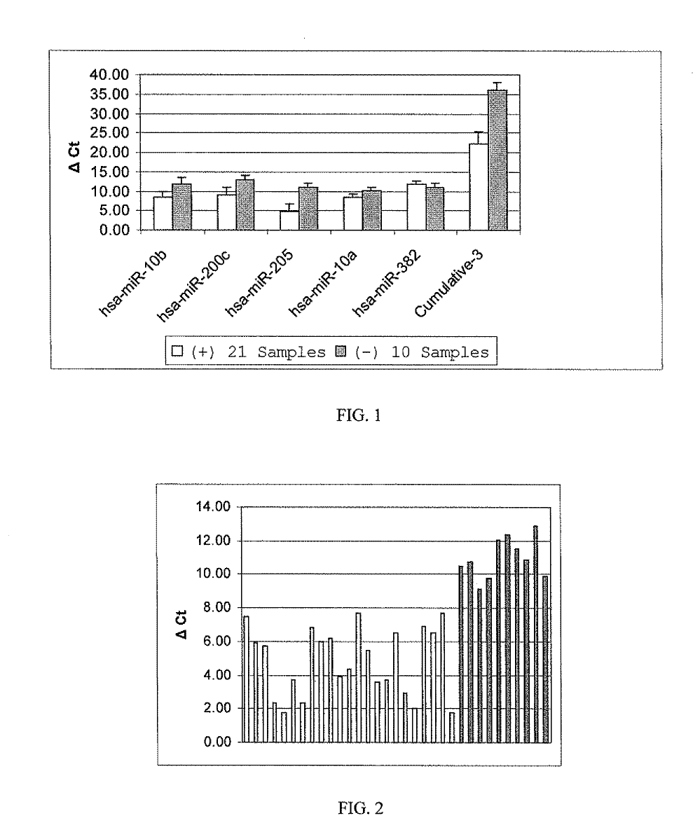

Markers that are associated with bioassay positive or negative samples were identified by a combination approach of gene microarrays and qRT-PCR. MicroRNA markers were evaluated by qRT-PCR and SYBR® Green detection using miRNA samples from cultured human dermal cell samples that were either positive or negative in inducing hair in conjunction with mouse neonatal epidermal cells in a bioassay (hybrid patch assay). Five markers that showed significant differences in expression between bioassay positive and negative samples and the data are shown in FIG. 1. The five markers are hsa-miR-10b, hsa-miR-200c, hsa-miR-205, hsa-miR-10a, and hsa-miR-382.

FIG. 1 is the graphical representation of average normalized Ct (ΔCt) values for each of the five miRNA markers assayed by qRT-PCR (quantitative real-time PCR) using SYBR®Green detection and miRNA from trichogenic (+) and non-trichogenic (−) dermal cell samples. Ct values are inversely proportional to expression of a gene and can be used to calculate the relative difference in expression of samples by using the formula: fold expression=2^{-ΔΔCt}, where ΔΔCt is the difference in normalized Ct values of the two samples being compared. The differences between the normalized Ct data of bioassay (+) and (−) samples for each marker are statistically significant as indicated by p values (<0.05) by Kruskal-Wallis test and ANOVA. The cumulative data for the three most distinguishing markers (hsa-miR-10b, hsa-miR-200c and hsa-miR-205) are also statistically significant between the bioassay (+) and (−) samples. Error bars are standard deviations.

### Example 4

### Variation of Biomarker Expression

Variation of biomarker expression among bioassay positive and negative samples for hsa-miR-205 is shown in FIG. 2. FIG. 2 shows the graphical representation of individual ΔCt values for hsa-miR-205 marker alone from trichogenic (+) and non-trichogenic (−) dermal cell samples. The average ΔCt±SD of (+) and (−) samples are (4.80±1.9) and (10.98±1.2) respectively. Hence the average fold difference in expression of the marker between bioassay (+) and (−) samples is 70 based on the difference in their average ΔCt values. The data are statistically significantly different between bioassay positive and negative samples as determined by Kruskal-Wallis test and ANOVA. All bioassay positive samples had higher expression (lower ΔCt values) in contrast to bioassay negative samples.

### Example 5

### Combined Biomarker Analysis

Cumulative normalized Ct values of hsa-miR-10b, hsa-miR-200c and hsa-miR-205 were used to analyze bioassay positive and negative samples in a combined fashion. The data are summarized in Tables 1. Table 1 displays cumulative ΔCt values of hsa-miR-10b, hsa-miR-200c and hsa-miR-205 between bioassay positive samples and negative samples.

TABLE 1
Table 1 Summary of descriptive statistics for
bioassay positive and negative samples.

| | Bioassay Positive | Bioassay Negative |
|---|---|---|
| Samples, n | 21 | 10 |
| Min | 15.23 | 30.32 |
| Max | 27.51 | 40.04 |
| Avg | 22.34 | 35.97 |
| Standard deviation | 3.08 | 1.93 |
| (Sd) | | |

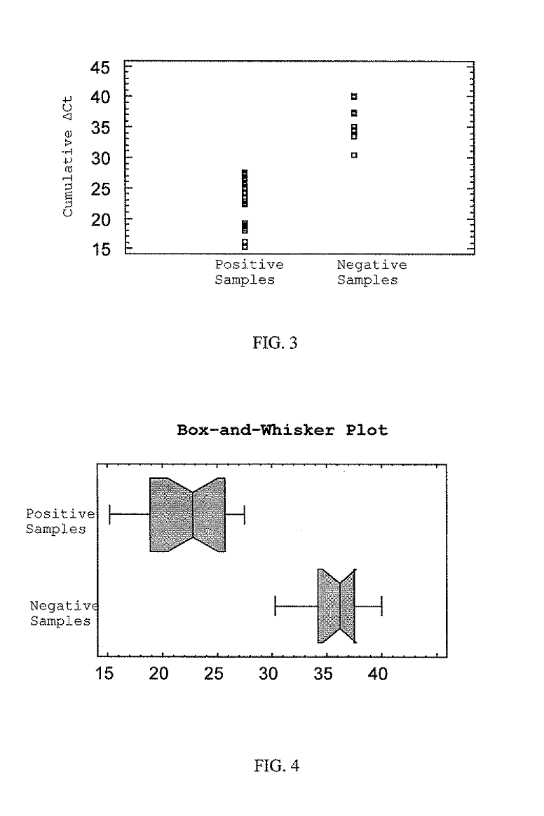

Spread of cumulative data for combined three markers data among bioassay positive and negative samples are shown in FIGS. 3 and 4. In this data-set there was no overlap in the data between bioassay positive and negative samples.

FIG. 3 shows a scatterplot of cumulative ΔCt values for three most distinguishing marker combination, hsa-miR-10b, hsa-miR-200c and hsa-miR-205, from 21 trichogenic (+) and 10 non-trichogenic (−) dermal cell samples. The average ΔCt±SD of (+) and (−) samples are (22.34±3.08) and (35.97±1.93) respectively. The data are statistically significantly different between bioassay positive and negative samples as determined by Kruskal-Wallis test and ANOVA.

FIG. 4 shows a Box and Whisker Plot of cumulative ΔCt values for hsa-miR-10b, hsa-miR-200c and hsa-miR-205 from 21 trichogenic (+) and 10 non-trichogenic (−) dermal cell samples. The spread of data is indicated by horizontal bars and the length of notch around the median (vertical bar) represents an approximate 95% CI for the median. No-overlap between the notches indicates that the data differ significantly.

### Example 5

### miRNA Markers for Trichogenicity

Another set of miRNA markers were identified using miRNA from bioassay positive samples (87) and bioassay negative samples (2) using qRT-PCR and Taqman® detection system. This set includes hsa-miR-200a, hsa-miR-200a*, hsa-miR-200a, hsa-miR-141, and hsa-miR-182. Bioassay positive samples (87) included 23 strongly positive and 64 moderately or weakly positive samples in bioassay.

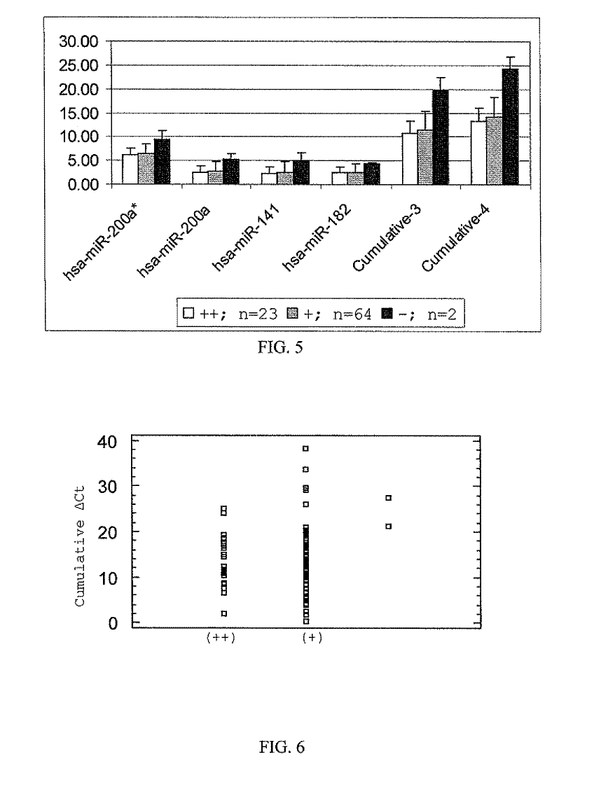

FIG. 5 shows a graphical representation of average normalized Ct (ΔCt) values (Y-axis) for hsamiR-200a*, hsa-miR-200a, hsa-miR-141, and hsa-miR-182 as well as cumulative (ΔCt) obtained from qRT-PCR (quantitative real-time PCR) using Taqman® detection system and miRNA from bioassay positive and bioassay negative dermal cell samples. Strongly bioassay positive samples (23 in number) are indicated by ++ and moderately/weakly positive (64 in number) are indicated by +. The differences between the normalized Ct data of bioassay (++) and (−) samples for each marker are statistically significant (p=<0.05), except hsa-miR-182 (p=0.057) as indicated Kruskal-Wallis test. There was no statistically significant difference between bioassay negative samples and moderately/weakly positive (+) samples for any of the four markers. The cumulative normalized Ct for all four markers are statistically significantly different between bioassay negative (−) and bioassay positive (+ or ++) samples (Kruskal-Wallis p=<0.05). On the other hand, cumulative normalized Ct for first three markers from left are statistically significantly different between bioassay negative (−) and bioassay positive (++) samples only (Kruskal-Wallis p=<0.05). Error bars are standard deviations.

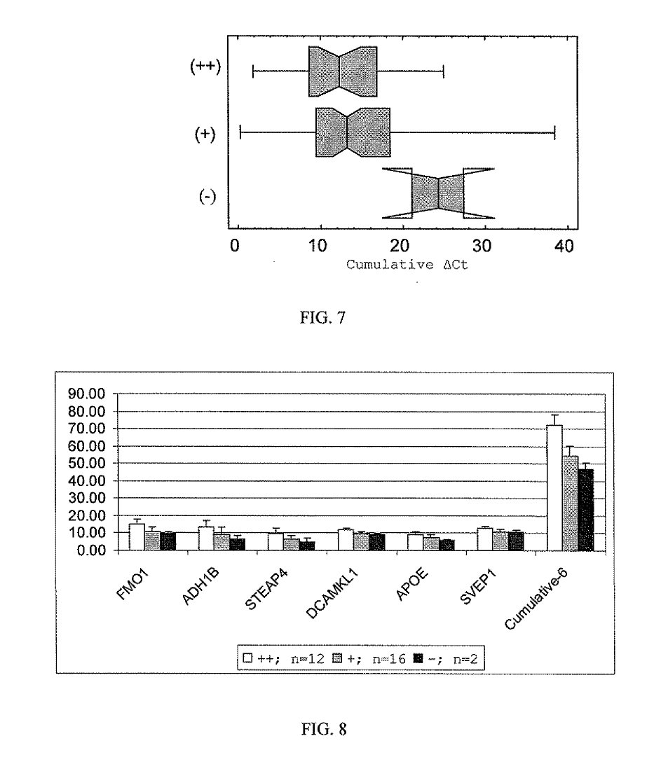

Spread of cumulative data for the four markers hsa-miR-200a*, hsa-miR-200a, hsa-miR-141 and hsa-miR-182 among bioassay positive and negative samples is shown in FIGS. 6 and 7. Although there was overlap in expression level between a few positive samples and the two negative samples, the vast majority of the positive samples did not overlap in their expression and the difference in expression of the four combined marker data are statistically significantly different between bioassay positive and negative samples.

FIG. 6 shows a scatterplot of cumulative ΔCt values for hsa-miR-141, hsa-miR-182, hsa-miR-200a and hsa-miR-200a* from 23 strongly positive (++), 64 moderately/weakly positive (+) and negative (−) dermal cell samples. The average ΔCt±SD of samples are: (++13.25±2.89), (+14.13 ±4.16) and (−24.26±2.57). The data are statistically significantly different between bioassay positive (++ or +) and negative (−) samples as determined by Kruskal-Wallis test (p=<0.05).

FIG. 7 shows a Box and Whisker Plot of cumulative ΔCt values for hsa-miR-141, hsa-miR-182, hsa-miR-200a and hsa-miR-200a* from 23 strongly positive (++), 64 moderately/weakly positive (+) and negative (−) dermal cell samples. The spread of data is indicated by horizontal bars and the length of notch around the median represents an approximate 95% CI for the median. Non-overlapping notches indicate that the two medians differ significantly.

Cumulative normalized Ct values of the four markers (hsa-miR-141, hsa-miR-182, hsa-miR-200a and hsa-miR-200a*) have been used to analyze bioassay positive and negative samples for three markers in a combined fashion. The data are summarized in Table 2.

TABLE 2
Table-2 Summary of descriptive statistics for
bioassay positive and negative samples.

| | Bioassay | Bioassay | Bioassay |
|---|---|---|---|
| | Positive (++) | Positive (+) | Negative |
| Samples, n | 23 | 64 | 2 |
| Min | 1.92 | 0.33 | 21.13 |
| Max | 24.93 | 38.35 | 27.38 |
| Avg | 13.25 | 14.13 | 24.26 |
| Standard deviation | 2.89 | 4.16 | 2.57 |
| (Sd) | | | |

### Example 6

### mRNA Biomarkers for Trichogenicity

Genes that are differentially expressed between trichogenic (bioassay positive) and non-trichogenic (bioassay negative) human cultured dermal cell samples were identified from microarray data of six independent cultured dermal samples. Markers that are either down-regulated or up-regulated in bioassay positive in contrast to bioassay negative samples were further characterized by qRT-PCR. See methods in Example 2. Several mRNA markers were confirmed by qRT-PCR and the oligonucleotide primers designed for qRT-PCR assay are shown in Tables 3 and 4. The data are summarized in FIGS. 8-13.

TABLE 3
Table 3. Dermal cell down-regulated mRNA markers and their DNA
oligonucleotide primer sequences used for RT-PCR.

| Gene | | | |
|---|---|---|---|
| Symbol | Gene Name | Forward Primer | Reverse Primer |
| FMO1 | Flavin containing | GCAAAACCCAACCTGTTCTC | GAGCATGGGCCAAAGAAGAC |
| | monooxygenase 1 | TATG | (SEQ ID NO: 2) |
| | | (SEQ ID NO: 1) | |
| ADH1B | Alcohol dehydrogenase | CCTGACGTTTTGAGGCAATAGA | CCTAGCTGTTGCTCCAGATCTTG |
| | 1B (class I) | (SEQ ID NO: 3) | (SEQ ID NO: 4) |
| STEAP4 | STEAP family member 4 | ACCTTTGGCCCCAACCA | GGGAAGGACAGAAGGAGAACTTG |
| | (TNF-alpha alpha- | (SEQ ID NO: 5) | (SEQ ID NO: 6) |
| | induced protein 9) | | |
| DCAMKL1 | Doublecortin-like kinase | ACCACAGCACAAAGTAACT | TCAACTAAGTCCATCAGACAGAGC |
| | 1 (Doublecortin and | GAACT | (SEQ ID NO: 8) |
| | CaM kinase like 1) | (SEQ ID NO: 7) | |
| APOE | Apoliopoprotein E | CCTTGGCCTGGCATCCT | GGAGCCGACTGGCCAAT |
| | | (SEQ ID NO: 9) | (SEQ ID NO: 10) |
| SVEP1 | Sushi, von Willebrand factor | GAATGCAGATTGGTTCTTCA | CGCCCAAATGCTTGTTCCT |
| | type A, EFG and pentraxin | CAGA | (SEQ ID NO: 12) |
| | domain containing 1 | (SEQ ID NO: 11) | |

TABLE 4
Table 4. Dermal cell up-regulated mRNA markers and their DNA
oligonucleotide primer sequences used for RT-PCR.

| Gene Symbol | Gene Name | Forward Primer Sequence | Reverse Primer Sequence |
|---|---|---|---|
| DEPDC1 | DEP domain containing | GGCGCTGACAGACCTATGGA | TGCTCGAAAAGATGTGGTAACTTC |
| (SDP35) | 1 (cell cycle control | (SEQ ID NO: 13) | (SEQ ID NO: 14) |
| | protein SDP35) | | |
| hFLEG1 | Fetal liver | CAGCGGCTGATAGAGAAGTA | GTAGGTCAGCGTGGCCATTT |
| (DJFZp-762E1312) | expressing gene 1 | CAAC | (SEQ ID NO: 16) |
| | | (SEQ ID NO: 15) | |
| ESM1 | Endothelial cell- | CGGTGGACTGCCCTCAAC | CGTCGAGCACTGTCCTCTTG |
| | specific molecule 1 | (SEQ ID NO: 17) | (SEQ ID NO: 18) |
| | (Endocan) | | |
| TOME-1 (CDCA3) | Trigger of mitotic | ATTGCACGGACACCTATGAAGA | CAGTTTCAAATACTTCACTCAGCTGTT |
| | entry 1 (cell | (SEQ ID NO: 19) | (SEQ ID NO: 20) |
| | division cycle | | |
| | associated 3) | | |
| THBD (CD141) | Thrombomodulin | TGTCCGCAGCGCTGTGT | GGTACTCGCAGTTGGCTCTGA |
| | (Fetomoldulin) | (SEQ ID NO: 21) | (SEQ ID NO: 22) |

Data of six mRNA markers that were identified to be down-regulated in bioassay positive samples in contrast to bioassay negative samples are shown in FIG. 8. Of the six markers FMO1, ADH1B, STEAP4, DCAMKL1, APOE, SVEP1, the three markers that showed maximum differences in average data between bioassay positive and negative samples are: FMO1, ADH1B and STEAP4.

FIG. 8 shows a graphical representation of average normalized Ct (ΔCt) values (Y-axis) for each of the six mRNA markers that are down-regulated in from bioassay positive dermal cells in contrast to bioassay negative cells as assayed by qRT-PCR (quantitative real-time PCR) using SYBR®Green detection system. Shown in the Figure is also cumulative (ΔCt) data from these six markers. Strongly positive samples (12 in number) are indicated by ++, moderately and weakly positive (16 in number) are indicated by +, and negative by −(2 in number). The differences between the normalized Ct data of bioassay (++) and (−) samples for the first five markers are statistically significant (p=<0.05) as indicated Kruskal-Wallis test. The sixth marker (SVEP1) is a weaker marker with Kruskal-Wallis p=0.0679. There was no statistically significant difference between bioassay negative samples and moderately/weakly positive (+) samples for any of the markers by the same test. The cumulative normalized Ct for all five markers are also statistically significantly different between bioassay negative (−) and bioassay positive (++) samples (Kruskal-Wallis p=<0.05) but not between bioassay negative (−) and bioassay weakly positive (+) samples by the same test. Error bars are standard deviations.

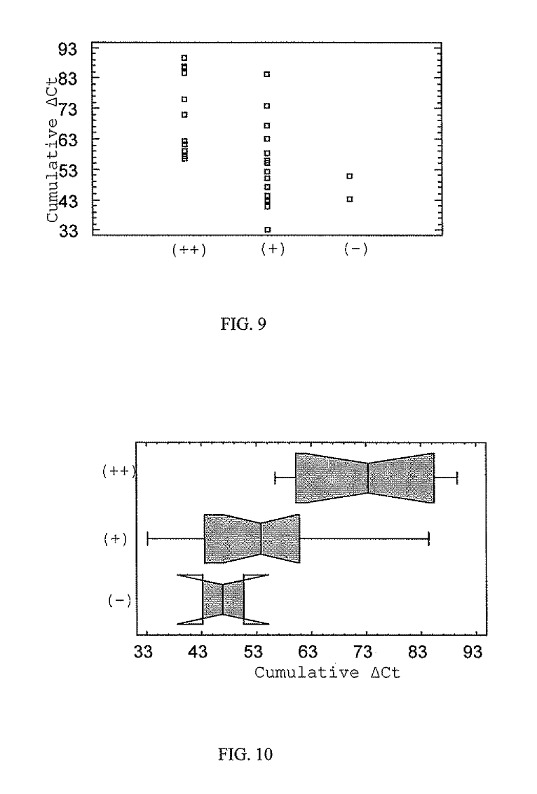

Spread of cumulative data for the five mRNA markers (down-regulated in bioassay positive dermal cells) among bioassay positive and negative samples is shown in FIGS. 9 and 10. Although there was overlap in expression data between few moderately/weakly positive (+) samples and the two negative samples, there was no overlap between bioassay strongly positive (++) and negative samples (−).

FIG. 9 shows a scatterplot of cumulative ΔCt values for six down-regulated mRNA markers from 12 strongly positive (++), 16 moderately/weakly positive (+) and 2 negative (−) dermal cell samples. The average cumulative ΔCt±SD of samples are: (++72.19±5.90), (+54.19±6.21) and (−46.88±3.75). The data are statistically significantly different between bioassay positive (++) and negative (−) samples as determined by Kruskal-Wallis test (p=<0.05).

FIG. 10 shows a Box and Whisker Plot of cumulative ΔCt values of six down-regulated mRNA markers from 12 strongly positive (++), 16 moderately/weakly positive (+) and 2 negative (−) dermal cell samples. The spread of data is indicated by horizontal bars and the length of notch around the median represents an approximate 95% CI for the median. Non-overlapping notches indicate that the two medians differ significantly.

### Example 7

### Up-Regulated Biomarkers in Bioassay Positive Dermal Cells

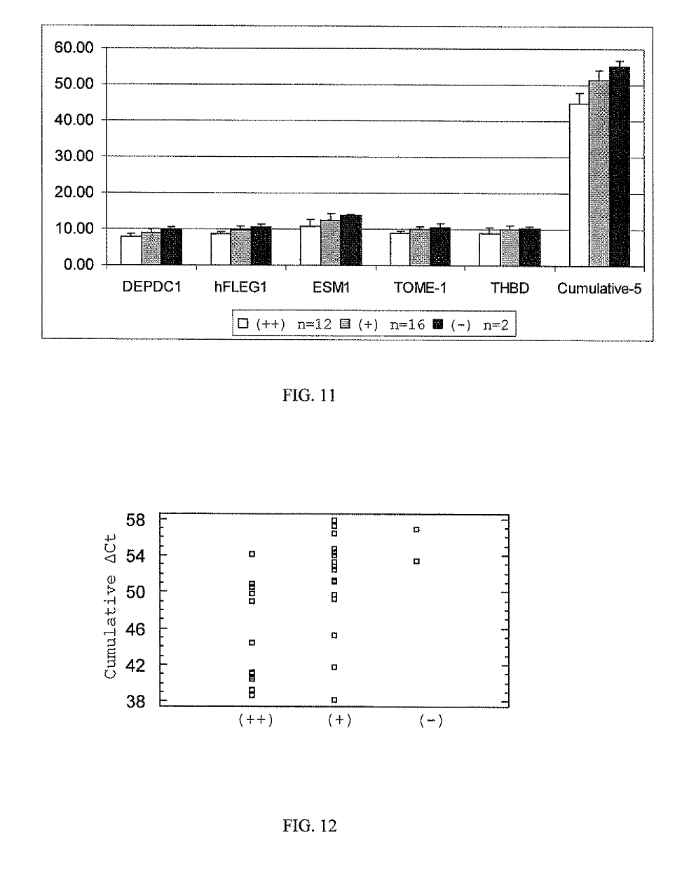

Five mRNA biomarkers were identified whose expression is up-regulated in bioassay positive samples in contrast to bioassay negative samples using the methods described in Example 2. These markers include DEPDC1, hFLEG1, ESM1, TOME-1, and THBD and their data are summarized in FIG. 11.

FIG. 11 shows a graphical representation of average normalized Ct (ΔCt) values (Y-axis) for each of the five mRNA markers that are up-regulated in mRNA from bioassay positive dermal cells in contrast to mRNA from bioassay negative dermal cells as assayed by qRT-PCR (quantitative real-time PCR) using SYBR®Green detection system. Also shown is cumulative (ΔCt) from these markers. Strongly positive samples (12 in number) are indicated by ++, moderately and weakly positive (16 in number) are indicated by +, and negative by −(2 in number). The differences between the normalized Ct data of bioassay (++) and (−) samples for each marker, except THBD are statistically significant (p=<0.05) as indicated Kruskal-Wallis test. There was no statistically significant difference between bioassay negative samples and moderately/weakly positive (+) samples for any of the markers by the same test. The cumulative normalized Ct for all five markers are also statistically significantly different between bioassay negative (−) and bioassay positive (++) samples (Kruskal-Wallis p=<0.05) but not between bioassay negative (−) and bioassay weakly positive (+) samples by the same test. Error bars are standard deviations.

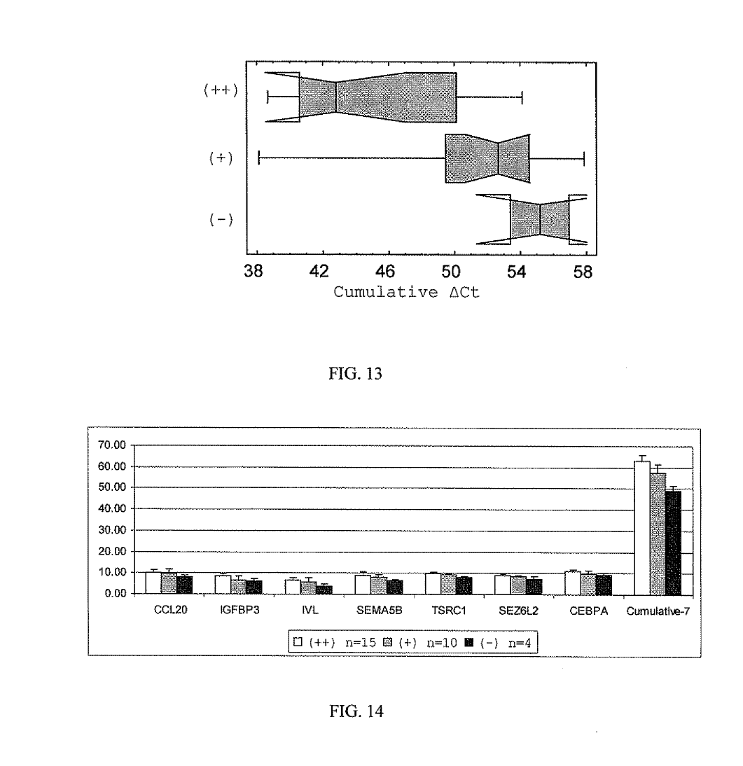

Spread of cumulative data for the five mRNA markers (down-regulated in bioassay positive dermal cells) among bioassay positive and negative samples are shown in FIGS. 12 and 13.

Although there was overlap in expression data between few moderately/weakly positive (+) samples and the two negative samples, with the exception of one sample, there was no overlap between bioassay strongly positive (++) and negative samples (−).

FIG. 13 shows a scatterplot of cumulative ΔCt values for five up-regulated mRNA markers from 12 strongly positive (++), 16 moderately/weakly positive (+) and 2 negative (−) dermal cell samples. The average cumulative ΔCt±SD of samples are: (++44.98±2.90), (+51.23±2.79) and (−55.19±1.64).

FIG. 14 shows a Box and Whisker Plot of cumulative ΔCt values of five up-regulated mRNA markers from 12 strongly positive (++), 16 moderately/weakly positive (+) and 2 negative (−) dermal cell samples. The spread of data is indicated by horizontal bars and the length of notch around the median represents an approximate 95% CI for the median. Non-overlapping notches indicate that the two medians differ significantly.

Additional genes whose expression differs significantly between trichogenic and non-trichogenic dermal cell samples are listed in Table 5.

TABLE 5
Table-5 Genes from gene microarray data whose expression
is either up-regulated or down-regulated.

| Category | Symbols of genes related to dermal cell trichogenicity |
|---|---|
| Up- | SFRS6, LOC400581, HNT, TNFRSF11B, FOSB, C5R1, HIST1H4C, |
| regulated | FGF5, MYBL1, FLJ20105, COL13A1, LOC134285, NEK2, TLR2, |
| | VEPH1, KIAA0179, ITGA8, STK6, USP13, C21orf56, CDC45L, |
| | C10orf3, TMSNB, TTK, PLAUR, CNIH3, DEPDC1B, ZFAND5, |
| | GALNT6, DKFZp313A2432, ASPM, EVI2A, ARTS-1, BUB1, NDP, |
| | CDC2, KIF11, HCAP-G, C20orf129, CYCS, TOB1, TBXA2R, |
| | FLJ11029, DLG7, KIAA1363, MGC34830, ATAD2, KIF4A, KNTC2, |
| | TYMS, KIAA0186, WHSC1, TMEM8, FLJ10038, C1GALT1, KCTD4, |
| | FUBP1, FL11, UBB, NSE1, PTPRD, TNFRSF21, CRYZ, |
| | DKFZp761D221, LOC283639, LIMD1, WNT5B, LOC157570, |
| | LOC401233, C1orf16, HNRPA1, INCENP, RNF175, CD47, RIN3, |
| | SEMA4B, OLFML1, EIF4G3, RoXaN, LRRN3, FZD1, LOC644246, |
| | CYYR1, LOC440820, ICK, EST1B, CYLD, PREX1, KIAA1462, MYO10, |
| | EIF2AK4, HHEX, HGF, LGR5, PTGIS, HRB2, EFHC2, STYK1, |
| | ST8SIA4, MYNN, PPP2R2C |
| Down- | DKFZP434P211, DKFZP434P211, SPOCK, PTGFR, PDE4DIP, |
| regulated | FOXO1A, FLJ14834, C9orf13, SERPING1, ABCA8, STXBP6, |
| | LOC339290, KCNE4, CXCL14, MMP10, IFI44L, SLC7A2, LIPG, |
| | SERPINA3, ACTG2, TMEM49, KIAA0746, TRIB3, DNM3, LOC440684 |
| | (LOC440886), EFEMP1, C5orf13, LOC401212, HCA112, ADAMTS2, |
| | GALNTL2, LOC654342, RASD1, SIX2, ZNF179, DSIPI, DCN, |
| | LOC283788, CDH2, SYTL4, ASNS, CDW92, HES4, RASGRP2, BET1L, |
| | CDK5RAP2, SOX4, AGRN, C12orf22, LIG3, PLEKHG2, NFATC1, |
| | LOC440885, RPL37A, SDCBP2, STRN3, SCRG1, NOTCH3, CTNNB1, |
| | C18orf11, GARP, SLC2A9, EPPK1, HRH1, C10orf47, JAG1, GABRE, |
| | RARRES1, HOXA2, GGA2, LOC158160, PCDH9, PCK2, KLF7, LU, |
| | AK3//AK3L2, LIN7B, COL12A1, INHBE, VSNL1, CES1, REC14, |
| | SUFU, MRPS11, RNF34, DKFZp667B0210, CACNB2, C13orf25 |

### Example 8

### Additional Dermal Cell Trichogenicity Markers Identified By Comparative Analysis To Mouse Trichogenic Cells

Genes that are differentially expressed between highly trichogenic non-cultured mouse neonatal dermal cells and cultured (Toma et al., Nat Cell Biol, 3:778-784 (2001)) but non-trichogenic neonatal mouse dermal cells were identified. Similarly, a differential gene profile of cultured adult mouse dermal cells that were either trichogenic or non-trichogenic were obtained. These gene profiles were compared with gene profiles of human data of trichogenic and non-trichogenic cells. Common genes that are potential candidate genes associated with the trichogenic activity of cells are listed in Table 6 and Table 7.

The list from Table 6 contains genes that by microarray data show 2-fold or more difference in expression in mouse trichogenic (neonatal dermal) vs non-trichogenic cells (cultured neonatal). The same genes also show a 1.5 fold or more difference in expression between trichogenic vs. non-trichogenic human dermal cell samples (p=<0.05). Interestingly, two genes from Table 5 (THBD and CDCA3) were identified independently by qRT-PCR evaluation of 30 cultured human dermal samples.

Tables 6A and 6B. Common genes in trichogenic mouse neonatal dermal cells and cultured human dermal cells.

TABLE 6A

| Class | Functional Category | Gene Symbols |
|---|---|---|
| Up- | Binding- ATP | MCM5, KIF23, MK167, EHD4, MPHOSPH1, |
| regulated | | KIAA0101, TOP2A |
| | Binding- Calcium | THBD |
| | Binding- GTP | GNB4, RAB11B, GSPT1 |
| | Binding- Hyaluronic | HMMR |
| | Acid | |
| | Binding- Nucleic | SFRS6, SFRS3, HNRNPA1, RAD51AP1, NUSAP1, |
| | acids | TMPO, PCNA, PRIM2 |
| | Binding- Protein | ALCAM, ROBO2, DTL, TM4SF1, CDCA3, CDCA8, |
| | | SPC25, CCNB1, CEP55, MAD2L1, SPC24, SPRY4, |
| | | TMEM158, TACC2, NCAPG2, SRGN, PMAIP1, |
| | | NCAPD3, C10RF71, DNAJC9, CD44, CENPF, |
| | | INCENP, STMN1 |
| | Cytoskeleton related | CDC42EP3, CKAP2, SORBS2, MTSS1, ANLN |
| | Enzyme | MARCH3, RRM2, FBXO5, POLE, TRDMT1 |
| | GPI anchor | CD55 |
| | GTPase activator | RGS20, RACGAP1, ARHGAP18 |
| | Guanyl-nucleotide | BCAR3, ECT2 |
| | Exchange Activity | |
| | Ion channel | KCTD12 |
| | Kinase | PBK, BUB1B, PFKFB3, BUB1, BCR, VRK1, TJP2 |
| | Peptidase Inhibitor | SERPINB5, |
| | Phosphatase | NT5E, DUSP10 |
| | Phosphatase Inhibitor | ANP32E |
| | Extracellular matrix | LMNB1 |
| | Transcription | Klf5, FOSB, ZNF367, E2F7, TRIP13, TOB1, EPC1, |
| | Regulator | TRIM24, EZH2, HMGB2 |
| | Transporter | UCP2, AP1S2 |
| | Unknown | COBLL1, LOC57228, GAS2L3, CKAP2L, RSRC1 |
| | | LOC130576, OLFML1, C130RF27, FAM33A, |
| | | TMCC3 |

TABLE 6B

| Class | Functional Category | Gene Symbols |
|---|---|---|
| Down- | Binding- ATP | TRIB3 |
| regulated | Binding- Calcium | SVEP1, NID1, COMMD5, LRP1, RCN3 |
| | Binding- GTP | RHOQ |
| | Binding- Nucleic | RBM6, BNC2, MBNL2, MRPS25, MRPL2, MCPH1, |
| | acids | RBMS2 |
| | Binding- Protein | MDM4, DOK1, IGFBP4, IFT122 |
| | Binding-other | C9ORF52, SELM, ZC3H11A, C18ORFJ7, LGALS8, |
| | molecules | LEPRE1 |
| | Cytokine | CCL2 |
| | Cytoskeleton related | CALD1 |
| | Enzyme | LOXL3, ASNS, CYP1B1, PCMTD1, CYB561, WARS, |
| | | AACS, AGPAT3, OGT, HEXA, ALDH1L2, FMO2, |
| | | GLT25D1, ALDH18A1 |
| | Extracellular matrix, | LAMA4, COL18A1, COL12A1 |
| | structural | |
| | GPI anchor | GPC1 |
| | GTPase activator | SIPA1L1 |
| | Ion channel | GRIA3, KCTD11 |
| | Kinase | DCLK1, PCK2, EGFR, CAMKK2, CDKN2B |
| | Peptidase | LGMN, ADAMTS15, BMP1, TAGLN |
| | Protease inhibitor | SERPINF1 |
| | Phosphatase | NUDT3 |
| | Receptor | EDG2, OSMR |
| | Transcription | SOX4, P8, KLF3, STAT2, GLIS3, DDIT3, RUNX1, RERE |
| | regulator | IF116, NFIA |
| | Transporter | BE1L, ATP6V0A2, PDPN, RSC1A1, LYST, C20ORF121 |
| | Translation | LOC387758 |
| | (initiation) | |
| | Unknown | TMEM176A, SRPX2, TNFAIP2, OLFML3, IQCE, |
| | | ORF19 |

The list from Table 7 contains genes that by microarray data show 2-fold or more difference in expression in adult mouse cultured trichogenic vs cultured non-trichogenic cells. Same genes also show 1.5 fold or more difference in expression between trichogenic vs. non-trichogenic human cultured dermal cell samples.

TABLE 7
Table-7 Common genes in trichogenic adult mouse cultured dermal cells
and cultured human dermal cells.

| Class | Functional Category | Gene Symbols |
|---|---|---|
| Up- | Binding- Nucleotides | EHD4, RFC2,ARL4C |
| regulated | Binding- Calcium | CCBE1 |
| | Binding- Cytoskeleton | SMTN |
| | Binding- Nucleic acids | SNRPA1 |
| | Binding- Protein | PMAIP1, SMG5, SRGN, |
| | | ALCAM, MEGF10 |
| | Enzyme | UBA6 |
| | GPI anchor | CDH13 |
| | GTPase activator | SIPA1L3 |
| | Ion channel | KCTD12, KCNN4 |
| | Kinase | TJP2 |
| | Peptidase | ARTS-1, ADAM8 |
| | Protease Inhibitor | PTTG1, SERPINB5 |
| | Phosphatase | PTPRF, NT5E, DUSP7, |
| | | DUSP10, DUSP4 |
| | Transcription Regulator | BNC1, TLE4, TBX3, |
| | | Klf5, BTBD11 |
| | Transporter | CSPG4 |
| | Unknown | C8ORF13, TMCC3 |
| Down- | Binding- ATP | TRIB3 |
| regulated | Binding- Calcium | SNED1, EFEMP1, C1S, |
| | | SVEP1, NID2, FBN1, |
| | | NID1 |
| | Binding- Nucleic acids | JHDM1D, ARID5B, |
| | | GPATCH2, RBMS3 |
| | Binding- Protein | GRB10, TXNIP, FLJ10324, |
| | | C4ORF34, VCL, PALLD, |
| | | MDM4, LRIG3 |
| | Cytokine | CXCL14 |
| | Cytoskeleton related | ADD3, CALD1, ACTG2 |
| | Enzyme | CYP1B1, ANAPC5, SULF1, |
| | | SULF2, GLT25D1 |
| | Extracellular matrix, | LAMA4, COL12A1 |
| | structural | |
| | GPI anchor | GPC1, GPC6 |
| | Growth Factor | FGF7 |
| | Ion channel | KCNE4 |
| | Kinase | AK3, PIK3C2A |
| | Peptidase | ADAMTS5, FAP, TAGLN |
| | Peptidase Inhibitor | |
| | Phosphatase | EYA4 |
| | Receptor | EDG2, TLR4, IL6ST, |
| | | OSMR, OLFML2A, VDR |
| | Transcription regulator | DDIT3, EBF1, SOX4, |
| | | SOX13, EMX2 |
| | Transporter | LYST |
| | Translation (initiation) | LOC387758 |
| | Unknown | C13ORF33, C9ORF150, |
| | | FAM20A, MCPH1, NDRG4, |
| | | LETMD1, FAM110B |

### Example 9

### Epidermal Cell Biomarkers For Trichogenicity

Genes that are differentially expressed between trichogenic (bioassay positive) and non-trichogenic (bioassay negative) human cultured epidermal cell samples were identified from microarray data of six independent cultured epidermal samples. Markers that are either down-regulated or up-regulated in bioassay positive in contrast to bioassay negative samples were further characterized by qRT-PCR. Several mRNA markers, that are down-regulated in trichogenic (bioassay positive) when compared to non-trichogenic (bioassay negative), were confirmed by qRT-PCR; the oligonucleotide primers designed for qRT-PCR assay for these seven mRNA markers are shown in Table 8. The data are summarized in FIGS. 14-16.

TABLE 8
Table 8. Epidermal cell markers (Down-regulated) and
their DNA oligonucleotide primer sequences used for RT-PCR.

| Gene Symbol | Gene Name | Forward Primer Sequence | Reverse Primer Sequence |
|---|---|---|---|
| CCL20 | Chemokine (C-C motif | AGTTGTCTGTGTGCGCAAATCC | ATGTGCAAGTGAAACCTCCAACCC |
| | ligand 20 | (SEQ ID NO: 23) | (SEQ ID NO: 24) |
| IGFBP3 | Insulin-like growth factor | TACAGTGCGCACAGGCTTTATCGAG | CGCCCTTGTTTCAGAAATGACACCAC |
| | binding protein 3 | (SEQ ID NO: 25) | (SEQ ID NO: 26) |
| IVL | Involucrin | AAATAACCACCCGCAGTGTCCAGA | GTAGAGGGACAGAGTCAAGTTCACAG |
| | | (SEQ ID NO: 27) | (SEQ ID NO: 28) |
| SEMA5B | Semaphorin 5B | AGCCTTGCCCTCAATGCACGAAA | AAGCAGGTCTCAGCCAACAACTCTGT |
| | | (SEQ ID NO: 29) | (SEQ ID NO: 30) |
| TSRC1 | ADAMTS-like 4 | TGTAACAGCCAACCCTGCAGCCA | ACATGTGCGCAAGAGCGGCAACA |
| | (thrombospondin repeat containing 1) | (SEQ ID NO: 31) | (SEQ ID NO: 32) |
| SEZ6L2 | Seizure related 6 homolog | AAACTGGAAGTGACCCAGACCACA | AGGGACTTTCCCTGAAGCTTGGTGTA |
| | (mouse)-like 2 | (SEQ ID NO: 33) | (SEQ ID NO : 34) |
| CEBPA | CCAAT/enhancer binding protein | TTGCCTAGGAACACGAAGCACGAT | CGCACATTCACATTGCACAAGGCACT |
| | (C/EBP), alpha | (SEQ ID NO: 35) | (SEQ ID NO: 36) |

Expression data from qRT-PCR of seven individual epidermal markers as well as cumulative data of the seven markers are shown in FIG. 14.

FIG. 14 shows a graphical representation of average normalized Ct (ΔCt) values (Y-axis) for each of the seven mRNA markers that are down-regulated in mRNA from bioassay positive cells in contrast to mRNA from bioassay negative cells as assayed by qRT-PCR (quantitative real-time PCR) using SYBR®Green detection system. The seven mRNA markers include CCL20, IGFBP3, IVL, SEMA5B, TSRC1, SEZ6L2, and CEBPA. Also shown is cumulative (ΔCt) from these seven markers. Strongly positive samples (15 in number) are indicated by (++), moderately and weakly positive (10 in number) are indicated by (+), and 4 negative by (−). The differences between the normalized Ct data of bioassay (++) and (−) samples for each marker are statistically significant (p=<0.05) as indicated Kruskal-Wallis test. There was also statistically significant difference (p=<0.05) between bioassay negative samples and moderately/weakly positive (+) samples for four markers (IVL, SEMA5B, TSRC1, SEZ6L2) by the same test. The cumulative normalized Ct for all seven markers are also statistically significantly different between bioassay negative (−) and bioassay positive (++ or +) samples (Kruskal-Wallis p=<0.05). Error bars are standard deviations.

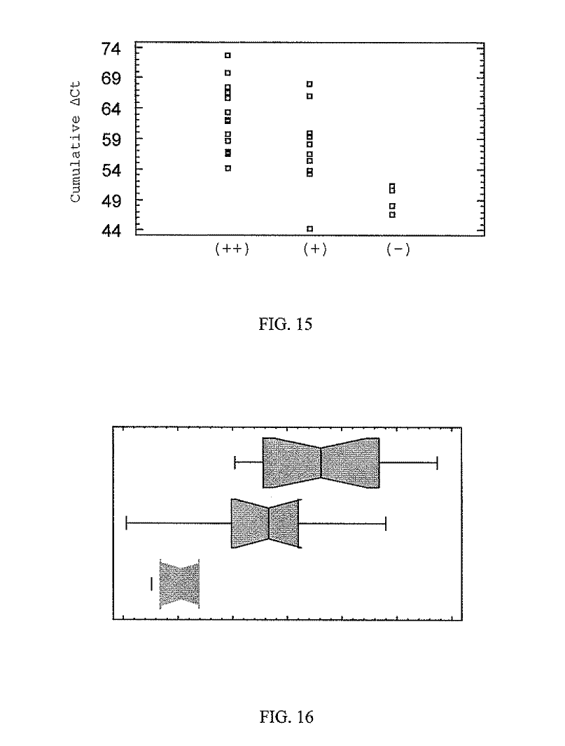

Spread of cumulative data for the seven mRNA markers (down-regulated in bioassay positive dermal cells) among bioassay positive and negative samples are shown in FIGS. 15 and 16. Except one moderately/weakly positive (+) sample there was no overlap in data between bioassay positive and negative samples.

FIG. 15 shows a scatterplot of cumulative ΔCt values for seven down-regulated mRNA markers (CCL20, IGFBP3, IVL, SEMA5B, TSRC1, SEZ6L2, and CEBPA) from 15 strongly positive (++), 10 moderately/weakly positive (+) and 4 negative (−) dermal cell samples. The average ΔCt±SD of samples are: (++62.96±2.91), (+57.51±3.98) and (−49.15±2.16).

FIG. 16 shows a Box and Whisker Not of cumulative ΔCt values seven down-regulated mRNA markers (CCL20, IGFBP3, IVL, SEMA5B, TSRC1, SEZ6L2, and CEBPA) from 15 strongly positive (++), 10 moderately/weakly positive (+) and 4 negative (−) dermal cell samples. The spread of data is indicated by horizontal bars and the length of notch around the median represents an approximate 95% CI for the median. Non-overlapping notches indicate that the two medians differ significantly.

Additional genes whose expression differs significantly between trichogenic and non-trichogenic epidermal cell samples are listed in Table 9.

TABLE 9
Table-9 Genes from gene microarray data whose expression is differentially regulated
(>2 fold, p value = <0.05) between trichogenic (bioassay positive, n = 3) and
non-trichogenic (bioassay negative, n = 3) cultured epidermal human samples.
Symbols of genes related to epidermal cell trichogenicity

APCDD1, IGFBP5, DKFZP586H2123, TXNIP, SCN4B, KRT15, MYLK,
PLAC2, UGT1A10//UGT1A8//UGT1A7, CXXC5 , GATA3, MAP2, MGC13102,
C6orf141, AQP3, DR1, DSC1, HOXA2 , ABHD6, RRAD, PPAP2C,
KIAA1644, NFATC1, AD023, MYLK, FOSL2, IHPK2, DOC1, KRT1,
CYP2S1, NOTCH3, LGALS7, ABLIM1, CBX4, EPHA4, MUC20, TAGLN,
SLC28A3, FOXC1, PVRL4, AMT, KCNJ5, MAF, KIFC2, LOC283970,
DLX3, IL1RN, THRA//NR1D1, TMC4, LOC401320, NIP, EPHB3, MYL9,
LOC388335, MARS, C9orf150, C9orf16, PRO1073, BIRC4BP, C5orf19,
ERBB3, P53AIP1, IL7, ZNF580, C110RF4, EPS8L1, DKFZP761M1511,
GAPDS, GGT1, TEAD3, FAM46B, BTG2, CEBPD, USP52, P8, MGC11335,
C2orf24, SYTL1, PKP1, PPT2, FOXO1A, ZNF606, EGf16, LOC284801,
GULP1, NSUN6, AVPR1B, BEX2, AKAP10, PIP5K1A, DUSP8, CXXC5,
ACBD4, MED12, MGC40489, MBNL1, IDUA, IL1R2, DAAM1, HIST1H2BG,
AADACL1, LPXN, ZFP42, MARCH4, MFAP5, MGC10850, ZNF367, RAB2,
MEST, RRM2, CYGB, C6orf62, HINT3, CLDN11, NPEPL1, ZBED2,
FEN1, ARHGAP18, DTL, NAV3, DUSP4, DHX29, LY6K, THBS1, DDAH1,
MYBL2, TNF, RAB12, CORO1A, ROBO4, ETV5, NRG1, SLC8A1,
HIST1H2BI, AMD1, CYP27B1, SLC39A8, Pfs2, CDC25A, NALP2, TAF1B,
DNMT2

1. A method for selecting trichogenic cells comprising assaying cells for expression of one or more biomarkers for trichogenicity; and selecting the cells having altered expression of the one or more biomarkers relative to a control.

2. The method of claim 1 wherein the one or more biomarkers are microRNA, mRNA, or protein.

3. The method of claim 1, wherein increased expression of the one or more biomarkers in the cells is indicative of trichogenicity, and the one or more biomarkers are encoded by a gene selected from the group consisting of hsa-miR-10b, hsa-miR-200c, hsa-miR-205, hsa-miR-10a and hsa-miR-382.

4. The method of claim 1, wherein increased expression of the one or more biomarkers in the cells is indicative of trichogenicity, and wherein one or more biomarkers are encoded by a gene selected from the group consisting of hsa-miR-200c, and hsa-miR-205.

5. The method of claim 1 further comprising the step of culturing the selected cells to increase the number of trichogenic cells.

6. The method of claim 1, wherein the cells are assayed for expression of at least two biomarkers wherein increased expression of the at least two biomarkers is indicative of trichogenicity.

7. The method of claim 1, wherein the cells are assayed for expression of at least three biomarkers wherein increased expression of the at least two biomarkers is indicative of trichogenicity.

8. The method of claim 1, wherein increased expression of the one or more biomarkers in the cells is indicative of trichogenicity, and wherein one or more biomarkers are encoded by a gene selected from the group consisting of hsa-miR-200a*, hsa-miR-200a, hsa-miR-141 and hsa-miR-182.

9. The method of claim 1, wherein increased expression of the one or more biomarkers in the cells is indicative of trichogenicity, and wherein the one or more biomarkers are encoded by a gene selected from the group consisting of DEPDC1, hFLEG1, ESM1, TOME-1 and THBD.

10. The method of claim 1, wherein increased expression of the one or more biomarkers in the cells is indicative of trichogenicity, wherein the one or more biomarkers are encoded by a gene selected from the group consisting of SFRS6, LOC400581, HNT, TNFRSF11B, FOSB, C5R1, HIST1H4C, FGF5, MYBL1, FLJ20105, COL13A1, LOC134285, NEK2, TLR2, VEPH1, KIAA0179, ITGA8, STK6, USP13, C21orf56, CDC45L, C10orf3, TMSNB, TTK, PLAUR, CNIH3, DEPDC1B, ZFAND5, GALNT6, DKFZp313A2432, ASPM, EVI2A, ARTS-1, BUB1, NDP, CDC2, KIF11, HCAP-G, C20orf129, CYCS, TOB1, TBXA2R, FLJ11029, DLG7, KIAA1363, MGC34830, ATAD2, KIF4A, KNTC2, TYMS, KIAA0186, WHSC1, TMEM8, FLJ10038, C1GALT1, KCTD4, FUBP1, FLI1, UBB, NSE1, PTPRD, TNFRSF21, CRYZ, DKFZp761D221, LOC283639, LIMD1, WNT5B, LOC157570, LOC401233, C1orf16, HNRPA1, INCENP, RNF175, CD47, RIN3, SEMA4B, OLFML1, EIF4G3, RoXaN, LRRN3, FZD1, LOC644246, CYYR1, LOC440820, ICK, EST1B, CYLD, PREX1, KIAA1462, MYO10, EIF2AK4, HHEX, HGF, LGR5, PTGIS, HRB2, EFHC2, STYK1, ST8SIA4, MYNN, and PPP2R2c.

11. The method of claim 1, wherein decreased expression of the biomarker in the cells is indicative of trichogenicity, wherein the biomarker is selected from the group consisting of FMO1, ADH1B, STEAP4, DCAMKL1 APOE and SVEP1.

12. The method of claim 1, wherein decreased expression of the biomarker in the cells is indicative of trichogenicity, wherein the biomarker is encoded by a gene selected from the group consisting of DKFZP434P211, DKFZP434P211, SPOOK, PTGFR, PDE4DIP, FOXO1A, FLJ14834, C9orf13, SERPING1, ABCA8, STXBP6, LOC339290, KCNE4, CXCL14, IFI44L, SLC7A2, LIPG, SERPINA3, ACTG2, TMEM49, KIAA0746, TRIB3, DNM3, LOC440684 (LOC440886), EFEMP1, C5orf13, LOC401212, HCA112, ADAMTS2, GALNTL2, LOC654342, RASD1, SIX2, ZNF179, DSIPI, DCN, LOC283788, CDH2, SYTL4, ASNS, CDW92, HES4, RASGRP2, BET1L, CDK5RAP2, SOX4, AGRN, C12orf22, LIG3, PLEKHG2, NFATC1, LOC440885, RPL37A, SDCBP2, STRN3, SCRG1, NOTCH3, CTNNB1, C18orf11, GARP, SLC2A9, EPPK1, HRH1, C10orf47, JAG1, GABRE, RARRES1, HOXA2, GGA2, LOC158160, PCDH9, PCK2, KLF7, LU, AK3//AK3L2, LIN7B, COL12A1, INHBE, VSNL1, CES1, REC14, SUFU, MRPS11, RNF34, DKFZp667B0210, and CACNB2, C13orf25.

13. The method of claim 1, wherein decreased expression of the biomarker in the cells is indicative of trichogenicity, wherein the biomarker is encoded by a gene selected from the group consisting of CCL20, IGFBP3, IVL, SEMA5B, TSRC1, SEZ6L2 and CEBPA.

14. A method to identify a compound for enhancing the hair-inducing capability of cultured cells comprising assaying the level of the biomarkers of claim 1 in the cells in presence and absence of the putative compound and selecting a compound that increases expression of biomarkers that are upregulated in trichogenic cells compared to non-trichogenic cells.

15. A method to enhance the trichogenic property of cells comprising inserting one or more nucleic acids encoding a biomarker encoded by one or more of the genes selected from the group consisting of hsa-miR-200a*, hsa-miR-200a, hsa-miR-141, hsa-miR-200c, hsa-miR-205, DEPDC1, hFLEG1, ESM1, TOME-1 and THBD or a combination thereof into cells obtained from a subject and selecting cells having increased expression of the biomarkers.

16. A method to suppress trichogenicity of cells comprising inserting one or more inhibitory nucleic acids that bind to mRNA of a biomarker for trichogenicity into cells obtained from a subject, wherein the biomarker is up-regulated in trichogenic cells relative to non-trichogenic cells.
